(19) United States
(12) Patent Application Publication
(10) Pub. No.: US 2025/0326782 A1
(43) Pub. Date: Oct. 23, 2025

(54) GLUCOSYLATED STEVIA COMPOSITIONS

(71) Applicant: PURECIRCLE SDN. BHD., NEGERI SEMBILAN (MY)

(72) Inventors: Avetik MARKOSYAN, Negeri Semilan (MY); Pei Chen KOH, Negeri Sembilan (MY); Yeen Yee WONG, Negeri Sembilan (MY); Siew Yin CHOW, Negeri Sembilan (MY); Khairul NIZAM BIN NAWI, Negeri Sembilan (MY)

(21) Appl. No.: 18/863,985

(22) PCT Filed: Apr. 28, 2023
(86) PCT No.: PCT/MY2023/050029

§ 371 Date: Nov. 7, 2024

(30) Foreign Application Priority Data
May 12, 2022 (MY) PI2022002489

(51) Int. Cl. C07H 15/203; A23L 5/20; A23L 27/20; A23L 29/00

(52) U.S. Cl. CPC C07H 15/203; A23L 5/27; A23L 27/2052; A23L 29/035

(57) ABSTRACT

Glucosylated chlorogenic acid (GCGA) compounds, particularly compounds selected from those having structural formulae (A), (B), (C), (D), (E), (F), salts thereof, and combinations thereof, and methods of preparing same are described. The GCGA compounds are useful in beverage products, food products, and home care products.

### TECHNICAL FIELD

The present invention relates to a process for preparing compositions comprising glucosylated Stevia compounds, including glucosylated chlorogenic acid compounds.

### BACKGROUND OF THE INVENTION

Phenolic compounds are secondary metabolites present naturally in abundant amounts in plants. As the term “phenol” suggests, the chemical substructure of phenolic compounds have aromatic ring(s) with one or more hydroxyl groups. Phenolic compounds are known to have antioxidant activities which are attributed to the capacity of scavenging free radicals, donating hydrogen atoms and electrons, or chelating metal cations. In the human body, phenolic compounds are important in defense responses, such as anti-aging, anti-inflammatory, antioxidant and anti-proliferative activities.

Chlorogenic acids are a group of phenolic compounds that consists of a quinic acid unit and hydroxycinnamic acid units, joined via ester linkages. Several hydroxycinnamic acids, such as caffeic acid, sinapic acid, ferulic acid, or p-coumaric acid are commonly found within chlorogenic acids as ester derivatives. Chlorogenic acids are one of the abundant polyphenols that can be found in coffee, fruits and vegetables. They are also found in the leaves of Stevia rebaudiana Bertoni, where the six major chlorogenic acid compounds are 3-O-caffeoyl quinic acid, 5-O-caffeoyl quinic acid, 4-O-caffeoyl quinic acid, 3,4-di-O-caffeoyl quinic acid, 4,5-di-O-caffeoyl quinic acid, and 3,5-di-O-caffeoyl quinic acid.

Despite the benefits of chlorogenic acid, its solubility in water is relatively low, at <2% (w/v). In addition, chlorogenic acid degrades at pH 2-4 over time, which limits its use in beverages that are usually prepared in acidic pH. There is limited information on other useful properties of these compounds. Accordingly, the need exists to increase solubility and stability of these compounds and identify new uses thereof.

### SUMMARY OF THE INVENTION

As used herein, the abbreviation term “CGA” refers to “chlorogenic acid(s)”. Both terms have the same meaning and may be used interchangeably and may refer to the singular form or the plural form.

As used herein, the term “CGA” or “chlorogenic acid(s)” refers to esters of quinic acid and caffeic acid, ferulic acid or p-coumaric acid, including, but not limited to, naturally occurring chlorogenic acids, e.g. 1-O-caffeoyl quinic acid, 3-O-caffeoyl quinic acid, 4-O-caffeoyl quinic acid, 5-O-caffeoyl quinic acid, 1,3-di-O-caffeoyl quinic acid, 1,4-di-O-caffeoyl quinic acid, 1,5-di-O-caffeoyl quinic acid, 3,4-di-O-caffeoyl quinic acid, 3,5-di-O-caffeoyl quinic acid, 4,5-di-O-caffeoyl quinic acid, 1,3,4-tri-O-caffeoyl quinic acid, 1,3,5-tri-O-caffeoyl quinic acid, 1,4,5-tri-O-caffeoyl quinic acid, 3,4,5-tri-O-caffeoyl quinic acid, 1,3,4,5-tetra-O-caffeoyl quinic acid, synthetic chlorogenic acids, salts thereof, and combinations thereof.

As used herein, the abbreviation term “GCGA” refers to “glucosylated chlorogenic acid(s)”. Both terms have the same meaning and may be used interchangeably and may refer to the singular form or the plural form. GCGA molecule may comprise at least one additional glucose molecule than CGA molecule. More specifically, GCGA molecule may comprise one additional glucose, two additional glucoses, three additional glucoses, four additional glucoses, five additional glucoses, six additional glucoses, seven additional glucoses, eight additional glucoses, nine additional glucoses or ten additional glucoses.

As used herein, the term “GCGA” or “glucosylated chlorogenic acid(s)” refers to glucoside or glucosides of chlorogenic acids, including, but not limited to, naturally occurring glucosylated chlorogenic acids, synthetic chlorogenic acids, e.g. enzymatically glucosylated chlorogenic acids, salts thereof, and combinations thereof.

As used herein, “biocatalysis” or “biocatalytic” refers to the use of natural or genetically engineered biocatalysts, such as enzymes, or cells comprising one or more enzyme, capable of single or multiple step chemical transformations on organic compounds. Biocatalysis processes include fermentation, biosynthesis, bioconversion and biotransformation processes. Both isolated enzymes, and whole-cell biocatalysis methods are known in the art. Biocatalyst protein enzymes can be naturally occurring or recombinant proteins.

The present invention provides a process for preparing a composition comprising a target GCGA, by contacting a starting composition comprising an organic substrate with a microbial cell and/or enzyme preparation, thereby producing a composition comprising a target GCGA.

The starting composition can comprise at least one compound selected from the group consisting of chlorogenic acids. In one embodiment, the starting composition is derived from Stevia rebaudiana Bertoni.

The target GCGA can be any GCGA. In one embodiment, the target GCGA is a synthetic GCGA.

In another embodiment, the target GCGA is compound A. The chemical structure of compound A is shown below.

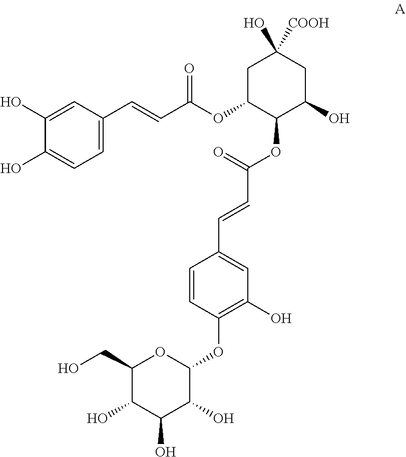 [SMILES: O=C(/C=C/c1ccc(O[C@H]2O[C@H](CO)[C@@H](O)[C@H](O)[C@H]2O)c(O)c1)O[C@@H]1[C@H](O)C[C@@](O)(C(=O)O)C[C@H]1OC(=O)/C=C/c1ccc(O)c(O)c1]

In another embodiment, the target GCGA is compound B. The chemical structure of compound B is shown below.

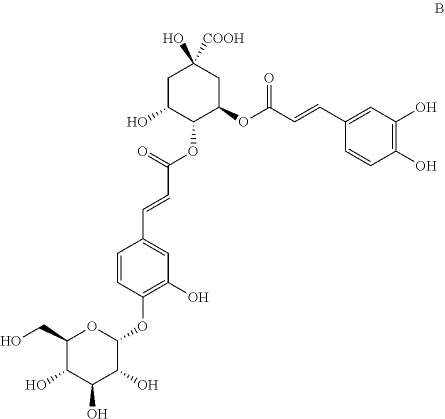 [SMILES: O=C(/C=C/c1ccc(O[C@H]2O[C@H](CO)[C@@H](O)[C@H](O)[C@H]2O)c(O)c1)O[C@@H]1[C@H](O)C[C@](O)(C(=O)O)C[C@H]1OC(=O)/C=C/c1ccc(O)c(O)c1]

In another embodiment, the target GCGA is compound C. The chemical structure of compound C is shown below.

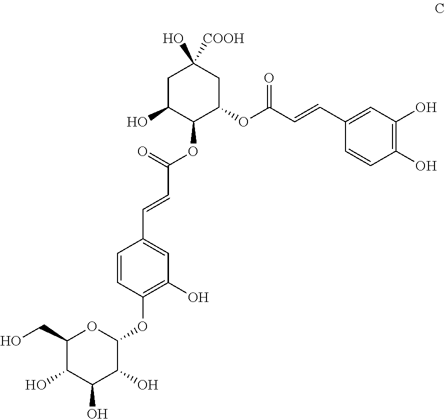 [SMILES: O=C(/C=C/c1ccc(O[C@H]2O[C@H](CO)[C@@H](O)[C@H](O)[C@H]2O)c(O)c1)O[C@@H]1[C@@H](OC(=O)/C=C/c2ccc(O)c(O)c2)C[C@@](O)(C(=O)O)C[C@@H]1O]

In another embodiment, the target GCGA is compound D. The chemical structure of compound D is shown below.

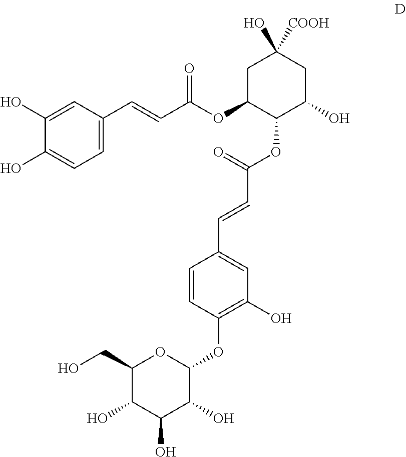 [SMILES: O=C(/C=C/c1ccc(O[C@H]2O[C@H](CO)[C@@H](O)[C@H](O)[C@H]2O)c(O)c1)O[C@@H]1[C@@H](OC(=O)/C=C/c2ccc(O)c(O)c2)C[C@](O)(C(=O)O)C[C@@H]1O]

In another embodiment, the target GCGA is compound E. The chemical structure of compound E is shown below.

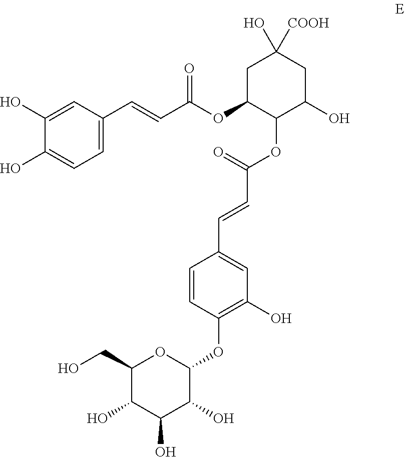 [SMILES: O=C(/C=C/c1ccc(O[C@H]2O[C@H](CO)[C@@H](O)[C@H](O)[C@H]2O)c(O)c1)OC1C(O)CC(O)(C(=O)O)C[C@@H]1OC(=O)/C=C/c1ccc(O)c(O)c1]

In another embodiment, the target GCGA is compound F. The chemical structure of compound F is shown below.

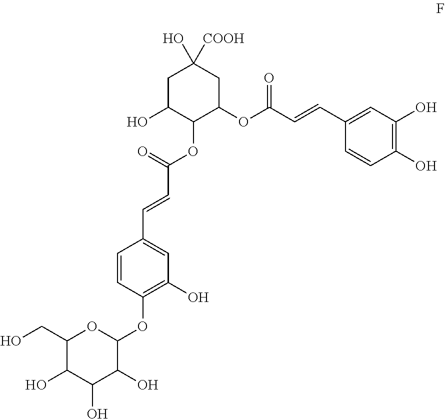 [SMILES: O=C(/C=C/c1ccc(O)c(O)c1)OC1CC(O)(C(=O)O)CC(O)C1OC(=O)/C=C/c1ccc(OC2OC(CO)C(O)C(O)C2O)c(O)c1]

In some preferred embodiments enzyme preparation comprising one or more enzymes, or a microbial cell comprising one or more enzymes, capable of converting the starting composition to target GCGA are used. The enzyme can be located on the surface and/or inside the cell. The enzyme preparation can be provided in the form of a whole cell suspension, a crude lysate or as purified enzyme(s). The enzyme preparation can be in free form or immobilized to a solid support made from inorganic or organic materials.

In some embodiments, a microbial cell comprises the necessary enzymes and genes encoding thereof for converting the starting composition to target GCGA. Accordingly, the present invention also provides a process for preparing a composition comprising a target GCGA by contacting a starting composition comprising an organic substrate with a microbial cell comprising at least one enzyme capable of converting the starting composition to target GCGA, thereby producing a medium comprising at least one target GCGA.

The enzymes necessary for converting the starting composition to target GCGA include, but not limited to, cyclodextrin glycosyltransferase, also known as cyclodextrin-glucanotransferase or cyclomaltodextrin glucanotransferase and abbreviated as CGTase. All four terms have the same meaning and may be used interchangeably.

The CGTase is any CGTase capable of adding at least one glucose unit to a starting CGA or GCGA bearing one or more-OH functional group to give a target GCGA having one or more additional-O-glucosyl glucopyranoside glycosidic linkage than the starting CGA or GCGA.

In another embodiment, the CGTase is any CGTase capable of adding at least one glucose unit to a starting CGA or GCGA bearing one or more-COOH functional group to give a target GCGA having one or more additional-COO-glucosyl glucopyranoside glycosidic linkage than the starting CGA or GCGA.

In one embodiment, the CGTase is present in one microorganism (microbial cell). The microorganism may be for example, Bacillus sp., Klebsiella pneumoniae, Micrococcus luteus, Thermococcus sp., Brevibacterium sp., Thermoactinomyces, hyperthermophilic archaea, Escherichia coli, Saccharomyces sp., Aspergillus sp., Pichia sp., and Yarrowia sp.

Optionally, the method of the present invention further comprises the use of other transglycosidases that use oligo- or poly-saccharides as the sugar donor to modify recipient CGA and/or GCGA compounds. Non-limiting examples include fructofuranosidase, amylase, dextransucrase, saccharase, glucosucrase, beta-h-fructosidase, beta-fructosidase, sucrase, fructosylinvertase. beta-fructofuranosidase, alkaline invertase and acid invertase. In some embodiments, glucose and sugar(s) other than glucose, including but not limited to fructose, xylose, rhamnose, arabinose, deoxyglucose, galactose are transferred to the recipient CGA and/or GCGA. In one embodiment, the recipient CGA is 1-O-caffeoyl quinic acid, 3-O-caffeoyl quinic acid, 4-O-caffeoyl quinic acid, 5-O-caffeoyl quinic acid, 1,3-di-O-caffeoyl quinic acid, 1,4-di-O-caffeoyl quinic acid, 1,5-di-O-caffeoyl quinic acid, 3,4-di-O-caffeoyl quinic acid, 3,5-di-O-caffeoyl quinic acid, 4,5-di-O-caffeoyl quinic acid, 1,3,4-tri-O-caffeoyl quinic acid, 1,3,5-tri-O-caffeoyl quinic acid, 1,4,5-tri-O-caffeoyl quinic acid, 3,4,5-tri-O-caffeoyl quinic acid or 1,3,4,5-tetra-O-caffeoyl quinic acid, salts thereof, or combinations thereof.

Optionally, the method of the present invention further comprises the use of NDP-glucosyltransferases (NGTs), ADP-glucosyltransferases (AGTs), CDP-glucosyltransferases (CGTs), GDP-glucosyltransferases (GGTs), TDP-glucosyltransferases (TDPs), UDP-glucosyltransferases (UGTs) to modify recipient CGA and/or GCGA compounds. Optionally it may include NDP-recycling enzyme(s), ADP-recycling enzyme(s), CDP-recycling enzyme(s), GDP-recycling enzyme(s), TDP-recycling enzyme(s), UDP-recycling enzyme(s) and/or sucrose synthase. In some embodiments, glucose and sugar(s) other than glucose, including but not limited to fructose, xylose, rhamnose, arabinose, deoxyglucose, galactose are transferred to the recipient CGA and/or GCGA. In one embodiment, the recipient CGA is 1-O-caffeoyl quinic acid, 3-O-caffeoyl quinic acid, 4-O-caffeoyl quinic acid, 5-O-caffeoyl quinic acid, 1,3-di-O-caffeoyl quinic acid, 1,4-di-O-caffeoyl quinic acid, 1,5-di-O-caffeoyl quinic acid, 3,4-di-O-caffeoyl quinic acid, 3,5-di-O-caffeoyl quinic acid, 4,5-di-O-caffeoyl quinic acid, 1,3,4-tri-O-caffeoyl quinic acid, 1,3,5-tri-O-caffeoyl quinic acid, 1,4,5-tri-O-caffeoyl quinic acid, 3,4,5-tri-O-caffeoyl quinic acid, or 1,3,4,5-tetra-O-caffeoyl quinic acid, salts thereof, or combinations thereof.

Optionally, the method of the present invention further comprises separating the target GCGA from the medium to provide a purified target GCGA composition. The target GCGA can be separated by at least one suitable method, such as, for example, crystallization, separation by membranes, centrifugation, extraction, chromatographic separation or a combination of such methods.

In one embodiment, the target GCGA can be produced within the microorganism. In another embodiment, the target GCGA can be secreted out in the medium. In one another embodiment, the released GCGA can be continuously removed from the medium. In yet another embodiment, the target GCGA is separated after the completion of the conversion reaction.

In one embodiment, separation produces a composition comprising greater than about 15% by weight of the target GCGA content on a dried basis. In another embodiment, separation produces a composition comprising greater than about 15%, greater than about 16%, greater than about 17%, greater than about 18%, greater than about 19%, greater than about 20%, greater than about 21%, greater than about 22%, greater than about 23%, greater than about 24%, greater than about 25%, greater than about 26%, greater than about 27%, greater than about 28%, greater than about 29%, greater than about 30%, greater than about 31%, greater than about 32%, greater than about 33%, greater than about 34%, greater than about 35%, greater than about 36%, greater than about 37%, greater than about 38%, greater than about 39%, greater than about 40%, greater than about 41%, greater than about 42%, greater than about 43%, greater than about 44%, greater than about 45%, greater than about 46%, greater than about 47%, greater than about 48%, greater than about 49%, greater than about 50%, greater than about 51%, greater than about 53%, greater than about 55%, greater than about 57%, greater than about 59%, greater than about 61%, greater than about 63%, greater than about 65%, greater than about 67%, greater than about 69%, greater than about 71%, greater than about 73%, greater than about 75%, greater than about 77%, greater than about 79%, greater than about 81%, greater than about 83%, greater than about 85%, greater than about 87%, greater than about 89%, greater than about 91%, greater than about 93%, greater than about 95%, greater than about 97%, or greater than about 99% target GCGA content on a dried basis. The content of target GCGA (e.g., compound A, compound B, compound C, compound D, compound E, or compound F) in the purified target GCGA composition can be any of the aforementioned amounts.

The target GCGA can be in any salt, polymorphic or amorphous forms, including salts, hydrates, solvates, anhydrous or combinations thereof.

Purified target GCGA can be used in consumable products as an antioxidant, preservative, color stabilizer, flavor stabilizer, flavor with modifying properties, foaming suppressor, solubility enhancing agent, fragrance modifier, fragrance stabilizer, fragrance enhancer and/or deodorizer. Suitable consumable products include, but are not limited to, food, beverages, pharmaceutical compositions, tobacco products, nutraceutical compositions, oral hygiene compositions, lipsticks and mouthwashes.

Purified target GCGA can also be used in home care products as an antioxidant, preservative, color stabilizer, foaming suppressor, solubility enhancing agent, fragrance modifier, fragrance stabilizer, fragrance enhancer and/or deodorizer. Suitable home care products include, but are not limited to, laundry detergents (powder, liquid and tablet), fabric conditioners, dishwashing detergents (liquid and tablet), hard floor and surface cleaners, glass cleaners, carpet cleaners, oven cleaners, air fresheners, car shampoos, toilet care products and insect-control products.

Purified target GCGA can also be used in personal care products as an antioxidant, preservative, color stabilizer, foaming suppressor, solubility enhancing agent, fragrance modifier, fragrance stabilizer, fragrance enhancer and/or deodorizer. Suitable personal care products include, but are not limited to, lotions, cosmetics, hair dyes, hair creams, face creams, body creams, deodorants, sunscreens, perfumes, toothpastes, bath soaps, hand soaps, shampoos, conditioners, hair masks, hair sprays, hair treatment oils, face cleansers, face masks, face moisturizers, acne creams, nail polishes, nail polish removers, shaving creams, shaving foams, shaving gels, after-shave lotions, after-shave balms, after-shave creams, after-shave fluids, shower gels, shower liquids, body sprays, anti-perspirants, skin balms, skin wipes, baby oils, baby colognes, baby shampoos, baby body shampoos, baby lotions, baby bath liquids, baby soaps, baby gel lotions, baby head-to-toe baths and baby powders.

### BRIEF DESCRIPTION OF THE DRAWINGS

FIG. 1 shows the biocatalytic production of compound A from 4,5-di-O-caffeoyl quinic acid using the enzyme CGTase.

FIG. 2a shows the HPLC chromatogram of Stevia CGA composition. The peak with retention time of 3.364 minutes corresponds to 5-O-caffeoyl quinic acid. The peak with retention time 5.340 minutes corresponds to 3-O-caffeoyl quinic acid. The peak at 5.571 minutes corresponds to 4-O-caffeoyl quinic acid. The peak at 18.550 minutes corresponds to 3,4-di-O-caffeoyl quinic acid. The peak at 19.461 minutes corresponds to 3,5-di-O-caffeoyl quinic acid. The peak at 22.485 minutes corresponds to 4,5-di-O-caffeoyl quinic acid.

FIG. 2b shows the HPLC chromatogram of the product of the biocatalytic production of GCGA from Stevia CGA composition. The peak at 16.667 minutes corresponds to compound A.

FIG. 2c shows the HPLC chromatogram of compound A after purification by HPLC. The peak with retention time of 15.912 minutes correspond to compound A.

FIG. 3a shows the MSD chromatogram of compound A.

FIG. 3b shows the mass spectrum of compound A.

FIG. 4a shows the pH stability of Stevia CGA composition and GCGA composition at pH 2 and room temperature.

FIG. 4b shows the pH stability of Stevia CGA composition and GCGA composition at pH 6 and room temperature.

FIG. 4c shows the pH stability of Stevia CGA composition and GCGA composition at pH 9 and room temperature.

FIG. 4d shows the pH stability of Stevia CGA composition and GCGA composition at pH 2 and 4° C.

FIG. 4e shows the pH stability of Stevia CGA composition and GCGA composition at pH 6 and 4° C.

FIG. 4f shows the pH stability of Stevia CGA composition and GCGA composition at pH 9 and 4° C.

FIG. 5a shows the pH stability of Stevia CGA composition and GCGA composition at pH 2 and room temperature.

FIG. 5b shows the pH stability Stevia CGA composition and GCGA composition at pH 2.5 and room temperature.

FIG. 5c shows the pH stability of Stevia CGA composition and GCGA composition at pH 3 and room temperature.

FIG. 5d shows the pH stability of Stevia CGA composition and GCGA composition at pH 3.5 and room temperature.

FIG. 5e shows the pH stability of Stevia CGA composition and GCGA composition at pH 4 and room temperature.

FIG. 5f shows the pH stability of Stevia CGA composition and GCGA composition at pH 2 and 4° C.

FIG. 5g shows the pH stability of Stevia CGA composition and GCGA composition at pH 2.5 and 4° C.

FIG. 5h shows the pH stability of Stevia CGA composition and GCGA composition at pH 3 and 4° C.

FIG. 5i shows the pH stability of Stevia CGA composition and GCGA composition at pH 3.5 and 4° C.

FIG. 5j shows the pH stability of Stevia CGA composition and GCGA composition at pH 4 and 4° C.

FIG. 6 shows the color stability of the blue pea flower extract solution at room temperature, in the presence and absence of GCGA composition.

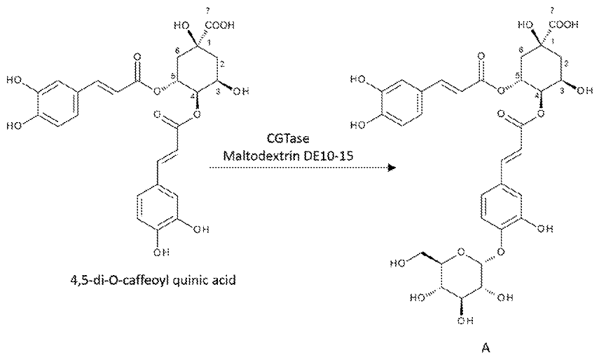

### DETAILED DESCRIPTION

The present invention provides a process for preparing a composition comprising a target GCGA by contacting a starting composition comprising an organic substrate with a microbial cell and/or enzyme preparation, thereby producing a composition comprising a target GCGA.

In an embodiment, a method for producing any of compound A, compound B, compound C, compound D, compound E and/or compound F includes the steps of providing a starting composition comprising at least one CGA compound and at least one sugar donor; providing an enzyme preparation or microorganism capable of adding at least one glucose unit to a starting CGA or GCGA bearing one or more —OH or —COOH functional group to give a target GCGA having one or more additional —O-glucosyl or —COO-glucosyl glucopyranoside glycosidic linkage than the starting CGA or GCGA; contacting the enzyme preparation or microorganism with a medium containing the starting composition to produce a medium comprising compound A, compound B, compound C, compound D, compound E, compound F, or combinations thereof.

Another object of the invention is to provide an efficient biocatalytic method for preparing target GCGA, particularly compound A, compound B, compound C, compound D, compound E, compound F and/or a synthetic GCGA from various starting compositions.

In an embodiment, a method for producing any of compound A, compound B, compound C, compound D, compound E and/or compound F includes the steps of providing a starting composition comprising at least one CGA compound and at least one sugar donor; providing a biocatalyst capable of adding at least one glucose unit to a starting CGA or GCGA bearing one or more —OH or —COOH functional group to give a target GCGA having one or more additional —O-glucosyl or —COO-glucosyl glucopyranoside glycosidic linkage than the starting CGA or GCGA; contacting the biocatalyst with a medium containing the starting composition to produce a medium comprising compound A, compound B, compound C, compound D, compound E, compound F, or combinations thereof.

Optionally, the methods above further include the step of separating or isolating compound A, compound B, compound C, compound D, compound E and/or compound F from the medium to provide a purified composition of compound A, compound B, compound C, compound D, compound E, compound F, or combinations thereof.

### Starting Composition

The starting composition can comprise at least one compound selected from the group consisting of chlorogenic acids.

In one embodiment, the starting composition is derived from Stevia rebaudiana Bertoni.

In another embodiment, the starting composition CGA is selected from the group consisting of 1-O-caffeoyl quinic acid, 3-O-caffeoyl quinic acid, 4-O-caffeoyl quinic acid, 5-O-caffeoyl quinic acid, 1,3-dicaffeoyl quinic acid, 1,4-di-O-caffeoyl quinic acid, 1,5-di-O-caffeoyl quinic acid, 3,4-di-O-caffeoyl quinic acid, 3,5-di-O-caffeoyl quinic acid, 4,5-di-O-caffeoyl quinic acid, 1,3,4-tri-O-caffeoyl quinic acid, 1,3,5-tri-O-caffeoyl quinic acid, 1,4,5-tri-O-caffeoyl quinic acid, 3,4,5-tri-O-caffeoyl quinic acid, 1,3,4,5-tetra-O-caffeoyl quinic acid, salts thereof, or combinations thereof.

In another embodiment, the starting composition is 4,5-di-O-caffeoyl quinic acid, or salts thereof.

The starting composition can also comprise at least one compound selected from the group consisting of GCGA or salts thereof, including but not limited to, synthetic GCGA or salts thereof, e.g. enzymatically glucosylated GCGA or salts thereof.

In one embodiment, the starting composition is compound A.

In yet another embodiment, the starting composition is compound B.

In yet another embodiment, the starting composition is compound C.

In yet another embodiment, the starting composition is compound D.

In yet another embodiment, the starting composition is compound E.

In yet another embodiment, the starting composition is compound F.

The starting composition may be synthetic or purified (partially or entirely), commercially available or prepared.

In one embodiment, the starting composition includes CGA.

In another embodiment, the starting composition further includes GCGA.

In another embodiment, the starting composition further includes maltodextrin.

In another embodiment, the starting composition further includes glucose.

In another embodiment, the starting composition further includes rhamnose.

In still another embodiment, the starting composition further includes sucrose.

In yet another embodiment, the starting composition further includes starch.

In yet another embodiment, the starting composition further includes at least one steviol glycoside.

In another embodiment, the starting composition further includes glycerol.

In yet another embodiment, the starting composition further includes cellulose.

In still another embodiment, the starting composition further includes amylose.

### Target GCGA

The target GCGA of the present method can be any GCGA that can be prepared by the process disclosed herein. GCGA compound may comprise at least one additional glucose molecule than CGA compound. More specifically, GCGA compound may comprise one additional glucose, two additional glucoses, three additional glucoses, four additional glucoses, five additional glucoses, six additional glucoses, seven additional glucoses, eight additional glucoses, nine additional glucoses or ten additional glucoses.

In one embodiment, the target GCGA is synthetic GCGA, e.g. enzymatically glucosylated GCGA.

In a particular embodiment, the target GCGA is compound A.

In a particular embodiment, the target GCGA is compound B.

In a particular embodiment, the target GCGA is compound C.

In a particular embodiment, the target GCGA is compound D.

In a particular embodiment, the target GCGA is compound E.

In a particular embodiment, the target GCGA is compound F.

The target GCGA can be in any salt, polymorphic or amorphous forms, including salts, hydrates, solvates, anhydrous or combinations thereof.

In one embodiment, the present invention is a biocatalytic process for the production of GCGA.

In another embodiment, the present invention is a biocatalytic process for the production of compound A. In a particular embodiment, the present invention provides for the biocatalytic process for the production of compound A from a starting composition comprising CGA and maltodextrin.

In another embodiment, the present invention is a biocatalytic process for the production of compound B. In a particular embodiment, the present invention provides for the biocatalytic process for the production of compound B from a starting composition comprising CGA and maltodextrin.

In another embodiment, the present invention is a biocatalytic process for the production of compound C. In a particular embodiment, the present invention provides for the biocatalytic process for the production of compound C from a starting composition comprising CGA and maltodextrin.

In another embodiment, the present invention is a biocatalytic process for the production of compound D. In a particular embodiment, the present invention provides for the biocatalytic process for the production of compound D from a starting composition comprising CGA and maltodextrin.

In another embodiment, the present invention is a biocatalytic process for the production of compound E. In a particular embodiment, the present invention provides for the biocatalytic process for the production of compound E from a starting composition comprising CGA and maltodextrin.

In another embodiment, the present invention is a biocatalytic process for the production of compound F. In a particular embodiment, the present invention provides for the biocatalytic process for the production of compound F from a starting composition comprising CGA and maltodextrin.

Optionally, the method of the present invention further comprises separating the target GCGA from the medium to provide a purified target GCGA composition. The target GCGA can be separated by any suitable method, such as, for example, crystallization, separation by membranes, centrifugation, extraction, chromatographic separation or a combination of such methods.

In particular embodiments, the process described herein results in a purified target GCGA composition. The term “purified”, as used herein, refers to a composition having greater than about 15% content by weight of the target GCGA on an anhydrous (dried) basis. In one embodiment, the purified target GCGA composition contains greater than about 15% content by weight of the target GCGA on an anhydrous (dried) basis, such as, for example, greater than about 15%, greater than about 16%, greater than about 17%, greater than about 18%, greater than about 19%, greater than about 20%, greater than about 21%, greater than about 22%, greater than about 23%, greater than about 24%, greater than about 25%, greater than about 26%, greater than about 27%, greater than about 28%, greater than about 29%, greater than about 30%, greater than about 31%, greater than about 32%, greater than about 33%, greater than about 34%, greater than about 35%, greater than about 36%, greater than about 37%, greater than about 38%, greater than about 39%, greater than about 40%, greater than about 41%, greater than about 42%, greater than about 43%, greater than about 44%, greater than about 45%, greater than about 46%, greater than about 47%, greater than about 48%, greater than about 49%, greater than about 50%, greater than about 51%, greater than about 53%, greater than about 55%, greater than about 57%, greater than about 59%, greater than about 61%, greater than about 63%, greater than about 65%, greater than about 67%, greater than about 69%, greater than about 71%, greater than about 73%, greater than about 75%, greater than about 77%, greater than about 79%, greater than about 81%, greater than about 83%, greater than about 85%, greater than about 87%, greater than about 89%, greater than about 91%, greater than about 93%, greater than about 95%, greater than about 97%, or greater than about 99% content by weight of the target GCGA on an anhydrous (dried) basis.

In one embodiment, when the target GCGA is compound A, the process described herein provides a composition having greater than about 15% compound A content by weight of the target GCGA on an anhydrous (dried) basis. In another particular embodiment, when the target GCGA is compound A, the process described herein provides a composition comprising greater than about 15% content by weight of the target GCGA on an anhydrous (dried) basis, such as, for example, greater than about 15%, greater than about 16%, greater than about 17%, greater than about 18%, greater than about 19%, greater than about 20%, greater than about 21%, greater than about 22%, greater than about 23%, greater than about 24%, greater than about 25%, greater than about 26%, greater than about 27%, greater than about 28%, greater than about 29%, greater than about 30%, greater than about 31%, greater than about 32%, greater than about 33%, greater than about 34%, greater than about 35%, greater than about 36%, greater than about 37%, greater than about 38%, greater than about 39%, greater than about 40%, greater than about 41%, greater than about 42%, greater than about 43%, greater than about 44%, greater than about 45%, greater than about 46%, greater than about 47%, greater than about 48%, greater than about 49%, greater than about 50%, greater than about 51%, greater than about 53%, greater than about 55%, greater than about 57%, greater than about 59%, greater than about 61%, greater than about 63%, greater than about 65%, greater than about 67%, greater than about 69%, greater than about 71%, greater than about 73%, greater than about 75%, greater than about 77%, greater than about 79%, greater than about 81%, greater than about 83%, greater than about 85%, greater than about 87%, greater than about 89%, greater than about 91%, greater than about 93%, greater than about 95%, greater than about 97%, or greater than about 99% content by weight of the target GCGA on an anhydrous (dried) basis.

In one embodiment, when the target GCGA is compound B, the process described herein provides a composition having greater than about 15% compound B content by weight of the target GCGA on an anhydrous (dried) basis. In another particular embodiment, when the target GCGA is compound B, the process described herein provides a composition comprising greater than about 15% content by weight of the target GCGA on an anhydrous (dried) basis, such as, for example, greater than about 15%, greater than about 16%, greater than about 17%, greater than about 18%, greater than about 19%, greater than about 20%, greater than about 21%, greater than about 22%, greater than about 23%, greater than about 24%, greater than about 25%, greater than about 26%, greater than about 27%, greater than about 28%, greater than about 29%, greater than about 30%, greater than about 31%, greater than about 32%, greater than about 33%, greater than about 34%, greater than about 35%, greater than about 36%, greater than about 37%, greater than about 38%, greater than about 39%, greater than about 40%, greater than about 41%, greater than about 42%, greater than about 43%, greater than about 44%, greater than about 45%, greater than about 46%, greater than about 47%, greater than about 48%, greater than about 49%, greater than about 50%, greater than about 51%, greater than about 53%, greater than about 55%, greater than about 57%, greater than about 59%, greater than about 61%, greater than about 63%, greater than about 65%, greater than about 67%, greater than about 69%, greater than about 71%, greater than about 73%, greater than about 75%, greater than about 77%, greater than about 79%, greater than about 81%, greater than about 83%, greater than about 85%, greater than about 87%, greater than about 89%, greater than about 91%, greater than about 93%, greater than about 95%, greater than about 97%, or greater than about 99% content by weight of the target GCGA on an anhydrous (dried) basis.

In one embodiment, when the target GCGA is compound C, the process described herein provides a composition having greater than about 15% compound C content by weight of the target GCGA on an anhydrous (dried) basis. In another particular embodiment, when the target GCGA is compound C, the process described herein provides a composition comprising greater than about 15% content by weight of the target GCGA on an anhydrous (dried) basis, such as, for example, greater than about 15%, greater than about 16%, greater than about 17%, greater than about 18%, greater than about 19%, greater than about 20%, greater than about 21%, greater than about 22%, greater than about 23%, greater than about 24%, greater than about 25%, greater than about 26%, greater than about 27%, greater than about 28%, greater than about 29%, greater than about 30%, greater than about 31%, greater than about 32%, greater than about 33%, greater than about 34%, greater than about 35%, greater than about 36%, greater than about 37%, greater than about 38%, greater than about 39%, greater than about 40%, greater than about 41%, greater than about 42%, greater than about 43%, greater than about 44%, greater than about 45%, greater than about 46%, greater than about 47%, greater than about 48%, greater than about 49%, greater than about 50%, greater than about 51%, greater than about 53%, greater than about 55%, greater than about 57%, greater than about 59%, greater than about 61%, greater than about 63%, greater than about 65%, greater than about 67%, greater than about 69%, greater than about 71%, greater than about 73%, greater than about 75%, greater than about 77%, greater than about 79%, greater than about 81%, greater than about 83%, greater than about 85%, greater than about 87%, greater than about 89%, greater than about 91%, greater than about 93%, greater than about 95%, greater than about 97%, or greater than about 99% content by weight of the target GCGA on an anhydrous (dried) basis.

In one embodiment, when the target GCGA is compound D, the process described herein provides a composition having greater than about 15% compound D content by weight of the target GCGA on an anhydrous (dried) basis. In another particular embodiment, when the target GCGA is compound D, the process described herein provides a composition comprising greater than about 15% content by weight of the target GCGA on an anhydrous (dried) basis, such as, for example, greater than about 15%, greater than about 16%, greater than about 17%, greater than about 18%, greater than about 19%, greater than about 20%, greater than about 21%, greater than about 22%, greater than about 23%, greater than about 24%, greater than about 25%, greater than about 26%, greater than about 27%, greater than about 28%, greater than about 29%, greater than about 30%, greater than about 31%, greater than about 32%, greater than about 33%, greater than about 34%, greater than about 35%, greater than about 36%, greater than about 37%, greater than about 38%, greater than about 39%, greater than about 40%, greater than about 41%, greater than about 42%, greater than about 43%, greater than about 44%, greater than about 45%, greater than about 46%, greater than about 47%, greater than about 48%, greater than about 49%, greater than about 50%, greater than about 51%, greater than about 53%, greater than about 55%, greater than about 57%, greater than about 59%, greater than about 61%, greater than about 63%, greater than about 65%, greater than about 67%, greater than about 69%, greater than about 71%, greater than about 73%, greater than about 75%, greater than about 77%, greater than about 79%, greater than about 81%, greater than about 83%, greater than about 85%, greater than about 87%, greater than about 89%, greater than about 91%, greater than about 93%, greater than about 95%, greater than about 97%, or greater than about 99% content by weight of the target GCGA on an anhydrous (dried) basis.

In one embodiment, when the target GCGA is compound E, the process described herein provides a composition having greater than about 15% compound E content by weight of the target GCGA on an anhydrous (dried) basis. In another particular embodiment, when the target GCGA is compound E, the process described herein provides a composition comprising greater than about 15% content by weight of the target GCGA on an anhydrous (dried) basis, such as, for example, greater than about 15%, greater than about 16%, greater than about 17%, greater than about 18%, greater than about 19%, greater than about 20%, greater than about 21%, greater than about 22%, greater than about 23%, greater than about 24%, greater than about 25%, greater than about 26%, greater than about 27%, greater than about 28%, greater than about 29%, greater than about 30%, greater than about 31%, greater than about 32%, greater than about 33%, greater than about 34%, greater than about 35%, greater than about 36%, greater than about 37%, greater than about 38%, greater than about 39%, greater than about 40%, greater than about 41%, greater than about 42%, greater than about 43%, greater than about 44%, greater than about 45%, greater than about 46%, greater than about 47%, greater than about 48%, greater than about 49%, greater than about 50%, greater than about 51%, greater than about 53%, greater than about 55%, greater than about 57%, greater than about 59%, greater than about 61%, greater than about 63%, greater than about 65%, greater than about 67%, greater than about 69%, greater than about 71%, greater than about 73%, greater than about 75%, greater than about 77%, greater than about 79%, greater than about 81%, greater than about 83%, greater than about 85%, greater than about 87%, greater than about 89%, greater than about 91%, greater than about 93%, greater than about 95%, greater than about 97%, or greater than about 99% content by weight of the target GCGA on an anhydrous (dried) basis.

In one embodiment, when the target GCGA is compound F, the process described herein provides a composition having greater than about 15% compound F content by weight of the target GCGA on an anhydrous (dried) basis. In another particular embodiment, when the target GCGA is compound F, the process described herein provides a composition comprising greater than about 15% content by weight of the target GCGA on an anhydrous (dried) basis, such as, for example, greater than about 15%, greater than about 16%, greater than about 17%, greater than about 18%, greater than about 19%, greater than about 20%, greater than about 21%, greater than about 22%, greater than about 23%, greater than about 24%, greater than about 25%, greater than about 26%, greater than about 27%, greater than about 28%, greater than about 29%, greater than about 30%, greater than about 31%, greater than about 32%, greater than about 33%, greater than about 34%, greater than about 35%, greater than about 36%, greater than about 37%, greater than about 38%, greater than about 39%, greater than about 40%, greater than about 41%, greater than about 42%, greater than about 43%, greater than about 44%, greater than about 45%, greater than about 46%, greater than about 47%, greater than about 48%, greater than about 49%, greater than about 50%, greater than about 51%, greater than about 53%, greater than about 55%, greater than about 57%, greater than about 59%, greater than about 61%, greater than about 63%, greater than about 65%, greater than about 67%, greater than about 69%, greater than about 71%, greater than about 73%, greater than about 75%, greater than about 77%, greater than about 79%, greater than about 81%, greater than about 83%, greater than about 85%, greater than about 87%, greater than about 89%, greater than about 91%, greater than about 93%, greater than about 95%, greater than about 97%, or greater than about 99% content by weight of the target GCGA on an anhydrous (dried) basis.

### Microorganisms and Enzyme Preparations

In one embodiment of present invention, a microorganism (microbial cell) and/or enzyme preparation is contacted with a medium containing the starting composition to produce target GCGA.

The enzyme can be provided in the form of a whole cell suspension, a crude lysate, a purified enzyme or a combination thereof. In one embodiment, the biocatalyst is a purified enzyme capable of converting the starting composition to the target GCGA. In another embodiment, the biocatalyst is a crude lysate comprising at least one enzyme capable of converting the starting composition to the target GCGA. In still another embodiment, the biocatalyst is a whole cell suspension comprising at least one enzyme capable of converting the starting composition to the target GCGA. In an embodiment, the biocatalyst is an enzyme, or a cell comprising one or more enzyme, capable of converting the starting composition to compound A, compound B, compound C, compound D, compound E, compound F, or combinations thereof.

In another embodiment, the biocatalyst is one or more microbial cells comprising enzyme(s) capable of converting the starting composition to the target GCGA. The enzyme can be located on the surface of the cell, inside the cell or located both on the surface of the cell and inside the cell.

Suitable enzymes for converting the starting composition to target GCGA include, but are not limited to CGTase, fructofuranosidase, amylase, dextransucrase, saccharase, glucosucrase, beta-h-fructosidase, beta-fructosidase, sucrase, fructosylinvertase, beta-fructofuranosidase, alkaline invertase and acid invertase, NDP-glucosyltransferases (NGTs), ADP-glucosyltransferases (AGTs), CDP-glucosyltransferases (CGTs), GDP-glucosyltransferases (GGTs), TDP-glucosyltransferases (TDPs), UDP-glucosyltransferases (UGTs). Optionally it may include NDP-recycling enzyme(s), ADP-recycling enzyme(s), CDP-recycling enzyme(s), GDP-recycling enzyme(s), TDP-recycling enzyme(s), UDP-recycling enzyme(s) and/or sucrose synthase.

The enzyme can be any enzyme capable of adding at least one glucose unit to CGA and/or a GCGA to provide the target GCGA. In a particular embodiment, the enzyme is CGTase.

In one embodiment, the enzyme is any enzyme capable of adding at least one glucose unit to a starting CGA or GCGA bearing one or more-OH functional group to give a target GCGA having one or more additional-O-glucosyl glucopyranoside glycosidic linkage than the starting CGA or GCGA. In another embodiment, the enzyme is any enzyme capable of adding two glucose units, three glucose units, four glucose units, five glucose units, six glucose units, seven glucose units, eight glucose units, nine glucose units or ten glucose units to a starting CGA or GCGA bearing one or more-OH functional group to give a target GCGA having two, three, four, five, six, seven, eight, nine or ten additional-O-glucosyl glucopyranoside glycosidic linkage than the starting CGA or GCGA. In a particular embodiment, the enzyme is CGTase.

In another embodiment, the enzyme is any enzyme capable of adding at least one glucose unit to a starting CGA or GCGA bearing one or more-COOH functional group to give a target GCGA having one or more additional-COO-glucosyl glucopyranoside glycosidic linkage than the starting CGA or GCGA. In another embodiment, the enzyme is any enzyme capable of adding two glucose units, three glucose units, four glucose units, five glucose units, six glucose units, seven glucose units, eight glucose units, nine glucose units or ten glucose units to a starting CGA or GCGA bearing one or more-COOH functional group to give a target GCGA having two, three, four, five, six, seven, eight, nine or ten additional-COO-glucosyl glucopyranoside glycosidic linkage than the starting CGA or GCGA. In a particular embodiment, the enzyme is CGTase.

In another embodiment, the enzyme is any CGTase capable of adding at least one glucose unit to CGA to form compound A.

In one embodiment, the enzyme is any CGTase capable of adding at least one glucose unit to 4,5-di-O-caffeoyl quinic acid to form compound A.

In another embodiment, the enzyme is any enzyme capable of adding at least one glucose unit to CGA to form compound B.

In another embodiment, the enzyme is any enzyme capable of adding at least one glucose unit to CGA to form compound C.

In another embodiment, the enzyme is any enzyme capable of adding at least one glucose unit to CGA to form compound D.

In another embodiment, the enzyme is any CGTase capable of adding at least one glucose unit to CGA to form compound E.

In another embodiment, the enzyme is any CGTase capable of adding at least one glucose unit to CGA to form compound F.

Optionally, the method of the present invention further comprises the use of transglycosidases that use oligo- or poly-saccharides as the sugar donor to modify recipient target GCGA compounds. Non-limiting examples include fructofuranosidase, amylase, dextransucrase, saccharase, glucosucrase, beta-h-fructosidase, beta-fructosidase, sucrase, fructosylinvertase, beta-fructofuranosidase, alkaline invertase and acid invertase. In some embodiments, glucose and sugar(s) other than glucose, including but not limited to fructose, xylose, rhamnose, arabinose, deoxyglucose, galactose are transferred to the recipient target CGA or GCGA. In one embodiment, the recipient GCGA is compound A. In other embodiments, the recipient GCGA is compound B, compound C, compound D, compound E, and/or compound F.

Optionally, the method of the present invention further comprises the use of NDP-glucosyltransferases (NGTs), ADP-glucosyltransferases (AGTs), CDP-glucosyltransferases (CGTs), GDP-glucosyltransferases (GGTs), TDP-glucosyltransferases (TDPs), UDP-glucosyltransferases (UGTs) to modify recipient target GCGA compounds. In some embodiments, glucose and sugar(s) other than glucose, including but not limited to fructose, xylose, rhamnose, arabinose, deoxyglucose, galactose are transferred to the recipient target CGA or GCGA. In some embodiments, NDP-recycling enzyme(s), ADP-recycling enzyme(s), CDP-recycling enzyme(s), GDP-recycling enzyme(s), TDP-recycling enzyme(s), UDP-recycling enzyme(s) and/or sucrose synthase. In one embodiment, the recipient GCGA is compound A. In other embodiments, the recipient GCGA is compound B, compound C, compound D, compound E, and/or compound F.

One embodiment of the present invention is a microbial cell comprising an enzyme, i.e. an enzyme capable of converting the starting composition to the target GCGA. Accordingly, some embodiments of the present method include contacting a microorganism with a medium containing the starting composition to provide a medium comprising at least one target GCGA.

The microorganism can be any microorganism possessing the necessary enzyme(s) for converting the starting composition to target GCGA. These enzymes are encoded within the microorganism's genome.

Suitable microorganisms include, but are not limited to, Bacillus sp., Klebsiella pneumoniae, Micrococcus luteus, Thermococcus sp., Brevibacterium sp., Thermoactinomyces, hyperthermophilic archaea, Escherichia coli, Saccharomyces sp., Aspergillus sp., Pichia sp., and Yarrowia sp.

In one embodiment, the microorganism is free when contacted with the starting composition.

In another embodiment, the microorganism is immobilized when contacted with the starting composition. For example, the microorganism may be immobilized to a solid support made from inorganic or organic materials. Non-limiting examples of solid supports suitable to immobilize the microorganism include derivatized cellulose or glass, ceramics, metal oxides or membranes. The microorganism may be immobilized to the solid support, for example, by covalent attachment, adsorption, cross-linking, entrapment or encapsulation.

In still another embodiment, the enzyme capable of converting the starting composition to the target GCGA is secreted out of the microorganism and into the reaction medium.

The target GCGA is optionally purified. Purification of the target GCGA from the reaction medium can be achieved by at least one suitable method to provide a purified target GCGA composition. Suitable methods include crystallization, separation by membranes, centrifugation, extraction (liquid or solid phase), chromatographic separation, HPLC (preparative or analytical) or a combination of such methods.

### Uses

Purified target GCGA, particularly compound A, compound B, compound C, compound D, compound E and/or compound F can be used “as-is” or in combination with other antioxidants, preservatives, color stabilizers, flavor stabilizers, flavors modifiers, flavors with modifying properties, foaming suppressors, solubility enhancing agents, fragrance modifiers, fragrance stabilizers, fragrance enhancers, deodorizers, sweeteners, food ingredients, home care product ingredients, personal care product ingredients, salts thereof, and combinations thereof.

Non-limiting examples of antioxidants include, but are not limited to, CGA, vitamin C, vitamin E, carotenoid, flavonols, flavanols, flavanones, flavones, isoflavonoids, anthocyanidins, glingerol, hydroxycinnamic acid, hydrobenzoic acid, curcumin, gallic acid, rosemary extract, rosmarinic acid. caffeic acid, carnosic acid, carnosol, butylated hydroxytoluene (BHT), butylated hydroxyanisole (BHA), tert-butylated hydroxyquinone (TBHQ), propyl 3,4,5-trihydroxybenzoate (PG), 1,2,3-trihydroxybenzene (Pyrogallol-PY), salts thereof, and combinations thereof.

Non-limiting examples of sweeteners include, but are not limited to, steviol glycosides, carbohydrates, psicose, tagatose, polyols, sugar alcohols, natural high intensity sweeteners, synthetic high intensity sweeteners, reduced calorie sweeteners, mogrosides, brazzein, neohesperidin dihydrochalcone, glycyrrhizic acid and its salts, thaumatin, perillartine, pernandulcin, mukuroziosides, baiyunoside, phlomisoside-I, dimethyl-hexahydrofluorene-dicarboxylic acid, abrusosides, periandrin, carnosiflosides, cyclocarioside, pterocaryosides, polypodoside A, brazilin, hernandulcin, phillodulcin, trilobatin, dihydroflavonol, dihydroquercetin-3-acetate, glycyphyllin, phlorizin, neoastilibin, trans-cinnamaldehyde, monatin, monatin salts, other indole derivative sweeteners, selligueain A, hematoxylin, monellin, osladin, pterocaryoside A, pterocaryoside B, mabinlin, pentadin, miraculin, curculin, neoculin, chlorogenic acid, cynarin, Luo Han Guo sweetener, mogroside V, siamenoside, siratose, salts thereof, and combinations thereof.

Non-limiting examples of flavors, flavor stabilizers, flavors with modifying properties include, but are not limited to, lime, lemon, orange, fruit, banana, grape, pear, pineapple, mango, berry, bitter almond, cola, cinnamon, sugar, cotton candy, vanilla, other compounds listed in FEMA (Flavor Extract Manufacturers Association) flavoring substances GRAS lists, salts thereof, and combinations thereof.

Non-limiting examples of other food ingredients include, but are not limited to, acidulants, organic and amino acids, coloring agents, bulking agents, modified starches, gums, texturizers, preservatives, caffeine, color stabilizers, flavor stabilizers, natural sweetener suppressors, additives, emulsifiers, stabilizers, thickeners, gelling agents, physiologically active substances, functional ingredients, salts thereof, and combinations thereof.

Purified target GCGA, particularly compound A, compound B, compound C, compound D, compound E and/or compound F can be prepared in any salt, polymorphic or amorphous forms, including but not limited to salts, hydrates, solvates, anhydrous, amorphous forms or combinations thereof.

Purified target GCGA, particularly compound A, compound B, compound C, compound D, compound E and/or compound F may be incorporated in foodstuffs, beverages, pharmaceutical compositions, cosmetics, chewing gums, tabletop products, cereals, dairy products, lipsticks, and toothpastes and other oral cavity compositions.

Purified target GCGA, particularly compound A, compound B, compound C, compound D, compound E and/or compound F may be incorporated in home care products, such as, but not limited to, laundry detergents (powder, liquid and tablet), fabric conditioners, dishwashing detergents (liquid and tablet), hard floor and surface cleaners, glass cleaners, carpet cleaners, oven cleaners, air fresheners, car shampoo, toilet care products, and insect-control products.

Purified target GCGA, particularly compound A, compound B, compound C, compound D, compound E and/or compound F may be incorporated in personal care products, such as, but not limited to, lotions, cosmetics, hair dyes, hair creams, face creams, body creams, deodorants, sunscreens, perfumes, toothpastes, bath soaps, hand soaps, shampoos, conditioners, hair masks, hair sprays, hair treatment oils, face cleansers, face masks, face moisturizers, acne creams, nail polishes, nail polish removers, shaving creams, shaving foams, shaving gels, after-shave lotions, after-shave balms, after-shave creams, after-shave fluids, shower gels, shower liquids, body sprays, anti-perspirants, skin balms, skin wipes, baby oils, baby colognes, baby shampoos, baby body shampoos, baby lotions, baby bath liquids, baby soaps, baby gel lotions, baby head-to-toe baths and baby powders.

Purified target GCGA, particularly compound A, compound B, compound C, compound D, compound E and/or compound F may be incorporated as an antioxidant in foodstuffs, beverages, pharmaceutical compositions, cosmetics, chewing gums, tabletop products, cereals, dairy products, toothpastes, other oral cavity compositions, home care products, and personal care products.

Purified target GCGA, particularly compound A, compound B, compound C, compound D, compound E and/or compound F may be incorporated as a preservative in foodstuffs, beverages, pharmaceutical compositions, cosmetics, chewing gums, tabletop products, cereals, dairy products, toothpastes, other oral cavity compositions, home care products, and personal care products.

Purified target GCGA, particularly compound A, compound B, compound C, compound D, compound E and/or compound F may be incorporated as a color stabilizer in foodstuffs, beverages, pharmaceutical compositions, cosmetics, chewing gums, tabletop products, cereals, dairy products, toothpastes, other oral cavity compositions, home care products, and personal care products.

Purified target GCGA, particularly compound A, compound B, compound C, compound D, compound E and/or compound F may be incorporated as a flavor stabilizer in foodstuffs, beverages, pharmaceutical compositions, cosmetics, chewing gums, tabletop products, cereals, dairy products, toothpastes, other oral cavity compositions, home care products, and personal care products.

Purified target GCGA, particularly compound A, compound B, compound C, compound D, compound E and/or compound F may be incorporated as a flavor with modifying properties in foodstuffs, beverages, pharmaceutical compositions, cosmetics, chewing gums, tabletop products, cereals, dairy products, toothpastes, other oral cavity compositions, home care products, and personal care products.

Purified target GCGA, particularly compound A, compound B, compound C, compound D, compound E and/or compound F may be incorporated as a foam stabilizer in foodstuffs, beverages, pharmaceutical compositions, cosmetics, chewing gums, table top products, cereals, dairy products, toothpastes, other oral cavity compositions, home care products, and personal care products.

Purified target GCGA, particularly compound A, compound B, compound C, compound D, compound E and/or compound F may be incorporated as a solubility enhancing agent in foodstuffs, beverages, pharmaceutical compositions, cosmetics, chewing gums, tabletop products, cereals, dairy products, toothpastes, other oral cavity compositions, home care products, and personal care products.

Purified target GCGA, particularly compound A, compound B, compound C, compound D, compound E and/or compound F may be incorporated as a fragrance modifier in foodstuffs, beverages, pharmaceutical compositions, cosmetics, chewing gums, tabletop products, cereals, dairy products, toothpastes, other oral cavity compositions, home care products, and personal care products.

Purified target GCGA, particularly compound A, compound B, compound C, compound D, compound E and/or compound F may be incorporated as a fragrance stabilizer in foodstuffs, beverages, pharmaceutical compositions, cosmetics, chewing gums, tabletop products, cereals, dairy products, toothpastes, other oral cavity compositions, home care products, and personal care products.

Purified target GCGA, particularly compound A, compound B, compound C, compound D, compound E and/or compound F may be incorporated as a fragrance enhancer in foodstuffs, beverages, pharmaceutical compositions, cosmetics, chewing gums, tabletop products, cereals, dairy products, toothpastes, other oral cavity compositions, home care products, and personal care products.

Purified target GCGA, particularly compound A, compound B, compound C, compound D, compound E and/or compound F may be incorporated as a deodorizer in foodstuffs, beverages, pharmaceutical compositions, cosmetics, chewing gums, tabletop products, cereals, dairy products, toothpastes, other oral cavity compositions, home care products, and personal care products.

In some embodiments, purified target GCGA of the present invention are present in consumable products, foodstuffs, beverages, pharmaceutical compositions, cosmetics, chewing gums, table top products, cereals, dairy products, toothpastes, other oral cavity compositions, alcoholic beverages such as vodka, wine, beer, liquor, and sake, natural juices, refreshing drinks, carbonated soft drinks, diet drinks, zero calorie drinks, reduced calorie drinks and foods, yogurt drinks, instant juices, instant coffee, powdered types of instant beverages, canned products, syrups, fermented soybean paste, soy sauce, vinegar, dressings, mayonnaise, ketchups, curry, soup, instant bouillon, powdered soy sauce, powdered vinegar, types of biscuits, rice biscuit, crackers, bread, chocolates, caramel, candy, chewing gum, jelly, pudding, preserved fruits and vegetables, fresh cream, jam, marmalade, flower paste, powdered milk, ice cream, sorbet, vegetables and fruits packed in bottles, canned and boiled beans, frozen beef, frozen pork, frozen goat, frozen lamb, frozen mutton, frozen poultry like frozen chicken, frozen duck and frozen turkey, frozen venison, frozen fish, frozen crustaceans like frozen crab and frozen lobster, frozen molluscs like frozen clams, frozen oysters, frozen scallops, and frozen mussels, frozen shrimps, frozen octopus, frozen squid, fresh beef, fresh pork, fresh goat, fresh lamb, fresh mutton, fresh poultry like fresh chicken, fresh duck and fresh turkey, fresh venison, fresh fish, fresh crustaceans like fresh crab and fresh lobster, fresh molluscs like fresh clams, fresh oysters, fresh scallops, and fresh mussels, fresh shrimps, fresh octopus, fresh squid, meat and foods boiled in sweetened sauce, agricultural vegetable food products, seafood, ham, sausage, fish ham, fish sausage, fish paste, deep fried fish products, dried seafood products, frozen food products, preserved seaweed, preserved meat, tobacco, lipsticks, and medicinal products in an amount from about 0.005% to about 0.5% by weight, such as, for example, about 0.005% by weight, about 0.01% by weight, about 0.02% by weight, about 0.03% by weight, about 0.04% by weight, about 0.05% by weight, about 0.06% by weight, about 0.07% by weight, about 0.08% by weight, about 0.09% by weight, about 0.10% by weight, about 0.11 by weight, about 0.12% by weight, about 0.13% by weight, about 0.14% by weight, about 0.15% by weight, about 0.16% by weight, about 0.17% by weight, about 0.18% by weight, about 0.19% by weight, about 0.20% by weight, about 0.21% by weight, about 0.22% by weight, about 0.23% by weight, about 0.24% by weight, about 0.25% by weight, about 0.26% by weight, about 0.27% by weight, about 0.28% by weight, about 0.29% by weight, about 0.30% by weight, about 0.31% by weight, about 0.32% by weight, about 0.33% by weight, about 0.34% by weight, about 0.35% by weight, about 0.36% by weight, about 0.37% by weight, about 0.38% by weight, about 0.39% by weight, about 0.40% by weight, about 0.41% by weight, about 0.42% by weight, about 0.43% by weight, about 0.44% by weight, about 0.45% by weight, about 0.46% by weight, about 0.47% by weight, about 0.48% by weight, about 0.49% by weight or about 0.5% by weight.

In some embodiments, purified target GCGA of the present invention are present in home care products, laundry detergents (powder, liquid and tablet), fabric conditioners, dishwashing detergents (liquid and tablet), hard floor and surface cleaners, glass cleaners, carpet cleaners, oven cleaners, air fresheners, car shampoo, toilet care products, and insect-control products in an amount from about 0.005% to about 0.5% by weight, such as, for example, about 0.005% by weight, about 0.01% by weight, about 0.02% by weight, about 0.03% by weight, about 0.04% by weight, about 0.05% by weight, about 0.06% by weight, about 0.07% by weight, about 0.08% by weight, about 0.09% by weight, about 0.10% by weight, about 0.11 by weight, about 0.12% by weight, about 0.13% by weight, about 0.14% by weight, about 0.15% by weight, about 0.16% by weight, about 0.17% by weight, about 0.18% by weight, about 0.19% by weight, about 0.20% by weight, about 0.21% by weight, about 0.22% by weight, about 0.23% by weight, about 0.24% by weight, about 0.25% by weight, about 0.26% by weight, about 0.27% by weight, about 0.28% by weight, about 0.29% by weight, about 0.30% by weight, about 0.31% by weight, about 0.32% by weight, about 0.33% by weight, about 0.34% by weight, about 0.35% by weight, about 0.36% by weight, about 0.37% by weight, about 0.38% by weight, about 0.39% by weight, about 0.40% by weight, about 0.41% by weight, about 0.42% by weight, about 0.43% by weight, about 0.44% by weight, about 0.45% by weight, about 0.46% by weight, about 0.47% by weight, about 0.48% by weight, about 0.49% by weight or about 0.5% by weight.

In some embodiments, purified target GCGA of the present invention are present in personal care products, lotions, cosmetics, hair dyes, hair creams, face creams, body creams, deodorants, sunscreens, perfumes, toothpastes, bath soaps, hand soaps, shampoos, conditioners, hair masks, hair sprays, hair treatment oils, face cleansers, face masks, face moisturizers, acne creams, nail polishes, nail polish removers, shaving creams, shaving foams, shaving gels, after-shave lotions, after-shave balms, after-shave creams, after-shave fluids, shower gels, shower liquids, body sprays, anti-perspirants, skin balms, skin wipes, baby oils, baby colognes, baby shampoos, baby body shampoos, baby lotions, baby bath liquids, baby soaps, baby gel lotions, baby head-to-toe baths and baby powders in an amount from about 0.005% to about 0.5% by weight, such as, for example, about 0.005% by weight, about 0.01% by weight, about 0.02% by weight, about 0.03% by weight, about 0.04% by weight, about 0.05% by weight, about 0.06% by weight, about 0.07% by weight, about 0.08% by weight, about 0.09% by weight, about 0.10% by weight, about 0.11 by weight, about 0.12% by weight, about 0.13% by weight, about 0.14% by weight, about 0.15% by weight, about 0.16% by weight, about 0.17% by weight, about 0.18% by weight, about 0.19% by weight, about 0.20% by weight, about 0.21% by weight, about 0.22% by weight, about 0.23% by weight, about 0.24% by weight, about 0.25% by weight, about 0.26% by weight, about 0.27% by weight, about 0.28% by weight, about 0.29% by weight, about 0.30% by weight, about 0.31% by weight, about 0.32% by weight, about 0.33% by weight, about 0.34% by weight, about 0.35% by weight, about 0.36% by weight, about 0.37% by weight, about 0.38% by weight, about 0.39% by weight, about 0.40% by weight, about 0.41% by weight, about 0.42% by weight, about 0.43% by weight, about 0.44% by weight, about 0.45% by weight, about 0.46% by weight, about 0.47% by weight, about 0.48% by weight, about 0.49% by weight or about 0.5% by weight.

In a particular embodiment, purified target GCGA of the present invention are present in the beverage in an amount from about 0.005% to about 0.5% by weight, such as, for example, about 0.005% by weight, about 0.01% by weight, about 0.02% by weight, about 0.03% by weight, about 0.04% by weight, about 0.05% by weight, about 0.06% by weight, about 0.07% by weight, about 0.08% by weight, about 0.09% by weight, about 0.10% by weight, about 0.11 by weight, about 0.12% by weight, about 0.13% by weight, about 0.14% by weight, about 0.15% by weight, about 0.16% by weight, about 0.17% by weight, about 0.18% by weight, about 0.19% by weight, about 0.20% by weight, about 0.21% by weight, about 0.22% by weight, about 0.23% by weight, about 0.24% by weight, about 0.25% by weight, about 0.26% by weight, about 0.27% by weight, about 0.28% by weight, about 0.29% by weight, about 0.30% by weight, about 0.31% by weight, about 0.32% by weight, about 0.33% by weight, about 0.34% by weight, about 0.35% by weight, about 0.36% by weight, about 0.37% by weight, about 0.38% by weight, about 0.39% by weight, about 0.40% by weight, about 0.41% by weight, about 0.42% by weight, about 0.43% by weight, about 0.44% by weight, about 0.45% by weight, about 0.46% by weight, about 0.47% by weight, about 0.48% by weight, about 0.49% by weight or about 0.5% by weight.

Purified target GCGA, particularly compound A, compound B, compound C, compound D, compound E and/or compound F, may be employed as an antioxidant compound, or it may be used together with at least one naturally occurring antioxidant compound such as vitamin C, vitamin E, carotenoid, flavonols, flavanols, flavanones, flavones, isoflavonoids, anthocyanidins, glingerol, hydroxycinnamic acid, hydrobenzoic acid, curcumin, gallic acid, rosemary extract, rosmarinic acid, caffeic acid, carnosic acid, carnosol, salts thereof and combinations thereof.

In a particular embodiment, purified target GCGA, particularly compound A, compound B, compound C, compound D, compound E and/or compound F can be used in an antioxidant composition comprising a compound selected from the group consisting of vitamin C, vitamin E, carotenoid, flavonols, flavanols, flavanones, flavones, isoflavonoids, anthocyanidins, glingerol, hydroxycinnamic acid, hydrobenzoic acid, curcumin, gallic acid, rosemary extract, rosmarinic acid, caffeic acid, carnosic acid, carnosol, salts thereof and combinations thereof.

Purified target GCGA, particularly compound A, compound B, compound C, compound D, compound E and/or compound F may also be used in combination with synthetic antioxidants such as butylated hydroxytoluene (BHT), butylated hydroxyanisole (BHA), tert-butylated hydroxyquinone (TBHQ), propyl 3,4,5-trihydroxybenzoate (PG), 1,2,3-trihydroxybenzene (Pyrogallol-PY), salts thereof, and combinations thereof.

Moreover, purified target GCGA particularly compound A, compound B, compound C, compound D, compound E and/or compound F can be used in combination with natural sweetener suppressors such as gymnemic acid, hodulcin, ziziphin, lactisole, and others. GCGA, particularly compound A, compound B, compound C, compound D, compound E and/or compound F may also be combined with various umami taste enhancers. GCGA, particularly compound A, compound B, compound C, compound D, compound E and/or compound F can be mixed with umami tasting and sweet amino acids such as aspartic acid, glycine, alanine, threonine, proline, serine, glutamate, lysine, tryptophan, salts thereof and combinations thereof.

Purified target GCGA, particularly compound A, compound B, compound C, compound D, compound E and/or compound F can be used in combination with one or more additive selected from the group consisting of carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, flavorants and flavoring ingredients, astringent compounds, proteins or protein hydrolysates, surfactants, emulsifiers, flavonoids, alcohols, polymers, salts thereof and combinations thereof.

Purified target GCGA, particularly compound A, compound B, compound C, compound D, compound E and/or compound F may be combined with polyols or sugar alcohols. The term “polyol” refers to a compound that contains more than one hydroxyl group. A polyol may be a diol, triol, or a tetraol which contain 2, 3, and 4 hydroxyl groups, respectively. A polyol also may contain more than four hydroxyl groups, such as a pentaol, hexaol, heptaol, or the like, which contain 5, 6, or 7 hydroxyl groups, respectively. Additionally, a polyol also may be a sugar alcohol, polyhydric alcohol, or polyalcohol which is a reduced form of carbohydrate, wherein the carbonyl group (aldehyde or ketone, reducing sugar) has been reduced to a primary or secondary hydroxyl group. Examples of polyols include, but are not limited to, erythritol, maltitol, mannitol, sorbitol, lactitol, xylitol, inositol, isomalt, propylene glycol, glycerol, threitol, galactitol, hydrogenated isomaltulose, reduced isomalto-oligosaccharides, reduced xylo-oligosaccharides, reduced gentio-oligosaccharides, reduced maltose syrup, reduced glucose syrup, hydrogenated starch hydrolyzates, polyglycitols and sugar alcohols or any other carbohydrates capable of being reduced which do not adversely affect the taste of the sweetener composition.

Purified target GCGA, particularly compound A, compound B, compound C, compound D, compound E and/or compound F may be combined with reduced calorie sweeteners such as, for example, psicose, D-tagatose, L-sugars, L-sorbose, L-arabinose and combinations thereof.

Purified target GCGA, particularly compound A, compound B, compound C, compound D, compound E and/or compound F may also be combined with various carbohydrates. The term “carbohydrate” generally refers to aldehyde or ketone compounds substituted with multiple hydroxyl groups, of the general formula (CH₂O)ₙ, wherein n is 3-30, as well as their oligomers and polymers. The carbohydrates of the present invention can, in addition, be substituted or deoxygenated at one or more positions. Carbohydrates, as used herein, encompass unmodified carbohydrates, carbohydrate derivatives, substituted carbohydrates, and modified carbohydrates. As used herein, the phrases “carbohydrate derivatives”, “substituted carbohydrate”, and “modified carbohydrates” are synonymous. Modified carbohydrate means any carbohydrate wherein at least one atom has been added, removed, or substituted, or combinations thereof. Thus, carbohydrate derivatives or substituted carbohydrates include substituted and unsubstituted monosaccharides, disaccharides, oligosaccharides, and polysaccharides. The carbohydrate derivatives or substituted carbohydrates optionally can be deoxygenated at any corresponding C-position, and/or substituted with one or more moieties such as hydrogen, halogen, haloalkyl, carboxyl, acyl, acyloxy, amino, amido, carboxyl derivatives, alkylamino, dialkylamino, arylamino, alkoxy, aryloxy, nitro, cyano, sulfo, mercapto, imino, sulfonyl, sulfenyl, sulfinyl, sulfamoyl, carboalkoxy, carboxamido, phosphonyl, phosphinyl, phosphoryl, phosphino, thioester, thioether, oximino, hydrazino, carbamyl, phospho, phosphonato, or any other viable functional group provided the carbohydrate derivative or substituted carbohydrate functions to improve the taste of the composition.

Examples of carbohydrates which may be used in accordance with this invention include, but are not limited to, psicose, turanose, allose, tagatose, trehalose, galactose, rhamnose, various cyclodextrins, cyclic oligosaccharides, various types of maltodextrins, dextran, sucrose, glucose, ribulose, fructose, threose, arabinose, xylose, lyxose, altrose, mannose, idose, lactose, maltose, invert sugar, isotrehalose, neotrehalose, isomaltulose, erythrose, deoxyribose, gulose, idose, talose, erythrulose, xylulose, cellobiose, amylopectin, glucosamine, mannosamine, fucose, glucuronic acid, gluconic acid, glucono-lactone, abequose, galactosamine, beet oligosaccharides, isomalto-oligosaccharides (isomaltose, isomaltotriose, panose and the like), xylo-oligosaccharides (xylotriose, xylobiose and the like), xylo-terminated oligosaccharides, gentio-oligosaccharides (gentiobiose, gentiotriose, gentiotetraose and the like), sorbose, nigero-oligosaccharides, palatinose oligosaccharides, fructooligosaccharides (kestose, nystose and the like), maltotetraol, maltotriol, malto-oligosaccharides (maltotriose, maltotetraose, maltopentaose, maltohexaose, maltoheptaose and the like), starch, inulin, inulo-oligosaccharides, lactulose, melibiose, raffinose, ribose, isomerized liquid sugars such as high fructose corn syrups, coupling sugars, and soybean oligosaccharides. Additionally, the carbohydrates as used herein may be in either the D- or L-configuration.

Purified target GCGA, particularly compound A, compound B, compound C, compound D, compound E and/or compound, can be used in combination with various physiologically active substances or functional ingredients. Functional ingredients generally are classified into categories such as carotenoids, dietary fiber, fatty acids, saponins, antioxidants, nutraceuticals, flavonoids, isothiocyanates, phenols, plant sterols and stanols (phytosterols and phytostanols), polyols, prebiotics, probiotics, phytoestrogens, soy protein, sulfides/thiols, amino acids, proteins, vitamins, and minerals. Functional ingredients also may be classified based on their health benefits, such as cardiovascular, cholesterol-reducing, and anti-inflammatory. Exemplary functional ingredients are provided in WO2013/096420, the contents of which is hereby incorporated by reference.

Purified target GCGA, particularly compound A, compound B, compound C, compound D, compound E and/or compound F, may be applied as an antioxidant to produce beverages, food products, home care products and personal care products with improved stability towards oxidation throughout storage compared to a control product that does not contain the compound. It may also be used in drinks, foodstuffs, pharmaceuticals, and other products in which oxidation minimization is preferred. In addition, purified target GCGA, particularly compound A, compound B, compound C, compound D, compound E and/or compound F can be used as an antioxidant not only for drinks, foodstuffs, home care products and personal care products and other products dedicated for human consumption, but also in animal feed and fodder with improved characteristics.

Purified target GCGA, particularly compound A, compound B, compound C, compound D, compound E and/or compound F, may be applied as a preservative to produce preserved beverages, food products, home care products and personal care products. It may also be used in drinks, foodstuffs, pharmaceuticals, and other products in which preservation is preferred. In addition, purified target GCGA, particularly compound A, compound B, compound C, compound D, compound E and/or compound F can be used as a preservative not only for drinks, foodstuffs, home care products and personal care products and other products dedicated for human consumption, but also in animal feed and fodder with improved characteristics.

Purified target GCGA, particularly compound A, compound B, compound C, compound D, compound E and/or compound F, may be applied as a color stabilizer to produce preserved beverages, food products, home care products and personal care products with improved color stability compared to a control product that does not contain the compound. It may also be used in drinks, foodstuffs, pharmaceuticals, and other products in which color stabilization is preferred. In addition, purified target GCGA, particularly compound A, compound B, compound C, compound D, compound E and/or compound F can be used as a color stabilizer not only for drinks, foodstuffs, home care products and personal care products and other products dedicated for human consumption, but also in animal feed and fodder with improved characteristics.

Purified target GCGA, particularly compound A, compound B, compound C, compound D, compound E and/or compound F, may be applied as a flavor stabilizer to produce preserved beverages, food products, home care products and personal care products with improved flavor stability compared to a control product that does not contain the compound. It may also be used in drinks, foodstuffs, pharmaceuticals, and other products in which flavor stabilization is preferred. In addition, purified target GCGA, particularly compound A, compound B, compound C, compound D, compound E and/or compound F can be used as a flavor stabilizer not only for drinks, foodstuffs, home care products and personal care products and other products dedicated for human consumption, but also in animal feed and fodder with improved characteristics.

Purified target GCGA, particularly compound A, compound B, compound C, compound D, compound E and/or compound F, may be applied as a flavor with modifying properties to produce preserved beverages, food products, home care products and personal care products with a modified flavor profile. It may also be used in drinks, foodstuffs, pharmaceuticals, and other products in which modifying a flavor profile is preferred. In addition, purified target GCGA, particularly compound A, compound B, compound C, compound D, compound E and/or compound F can be used as a flavor with modifying properties not only for drinks, foodstuffs, home care products and personal care products and other products dedicated for human consumption, but also in animal feed and fodder with improved characteristics.

Purified target GCGA, particularly compound A, compound B, compound C, compound D, compound E and/or compound F, may be applied as a foaming suppressor to produce preserved beverages, food products, home care products and personal care products with suppressed foaming. It may also be used in drinks, foodstuffs, pharmaceuticals, and other products in which foaming suppression is preferred. In addition, purified target GCGA, particularly compound A, compound B, compound C, compound D, compound E and/or compound F can be used as a foaming suppressor not only for drinks, foodstuffs, home care products and personal care products and other products dedicated for human consumption, but also in animal feed and fodder with improved characteristics.

Purified target GCGA, particularly compound A, compound B, compound C, compound D, compound E and/or compound F, may be applied as a solubility enhancing agent to produce preserved beverages, food products, home care products and personal care products having less insoluble material compared to a control product that does not contain the compound. It may also be used in drinks, foodstuffs, pharmaceuticals, and other products in which solubility enhancement is preferred. In addition, purified target GCGA, particularly compound A, compound B, compound C, compound D, compound E and/or compound F can be used as a solubility enhancing agent not only for drinks, foodstuffs, home care products and personal care products and other products dedicated for human consumption, but also in animal feed and fodder with improved characteristics.

Purified target GCGA, particularly compound A, compound B, compound C, compound D, compound E and/or compound F, may be applied as a fragrance modifier to produce preserved beverages, food products, home care products and personal care products having a modified fragrance compared to a control product that does not contain the compound. It may also be used in drinks, foodstuffs, pharmaceuticals, and other products in which fragrance modification is preferred. In addition, purified target GCGA, particularly compound A, compound B, compound C, compound D, compound E and/or compound F can be used as a fragrance modifier not only for drinks, foodstuffs, home care products and personal care products and other products dedicated for human consumption, but also in animal feed and fodder with improved characteristics.

Purified target GCGA, particularly compound A, compound B, compound C, compound D, compound E and/or compound F, may be applied as a fragrance stabilizer to produce preserved beverages, food products, home care products and personal care products with enhanced fragrance stability compared to a control product that does not contain the compound. It may also be used in drinks, foodstuffs, pharmaceuticals, and other products in which fragrance stabilization is preferred. In addition, purified target GCGA, particularly compound A, compound B, compound C, compound D, compound E and/or compound F can be used as a fragrance stabilizer not only for drinks, foodstuffs, home care products and personal care products and other products dedicated for human consumption, but also in animal feed and fodder with improved characteristics.

Purified target GCGA, particularly compound A, compound B, compound C, compound D, compound E and/or compound F, may be applied as a fragrance enhancer to produce preserved beverages, food products, home care products and personal care products with enhanced fragrance compared to a control product that does not contain the compound. It may also be used in drinks, foodstuffs, pharmaceuticals, and other products in which fragrance enhancement is preferred. In addition, purified target GCGA, particularly compound A, compound B, compound C, compound D, compound E and/or compound F can be used as a fragrance enhancer not only for drinks, foodstuffs, home care products and personal care products and other products dedicated for human consumption, but also in animal feed and fodder with improved characteristics.

Purified target GCGA, particularly compound A, compound B, compound C, compound D, compound E and/or compound F, may be applied as a deodorizer to produce preserved beverages, food products, home care products and personal care products with less or no odor compared to a control product that does not contain the compound. It may also be used in drinks, foodstuffs, pharmaceuticals, and other products in which deodorization is preferred. In addition, purified target GCGA, particularly compound A, compound B, compound C, compound D, compound E and/or compound F can be used as a deodorizer not only for drinks, foodstuffs, home care products and personal care products and other products dedicated for human consumption, but also in animal feed and fodder with improved characteristics.

Examples of consumable products in which purified target GCGA, particularly compound A, compound B, compound C, compound D, compound E and/or compound F may be used as an antioxidant, preservative, color stabilizer, flavor stabilizer, flavor with modifying properties, foaming suppressor, solubility enhancing agent, fragrance modifier, fragrance stabilizer, fragrance enhancer and/or deodorizer compound include, but are not limited to, alcoholic beverages such as vodka, wine, beer, liquor, and sake, natural juices, refreshing drinks, carbonated soft drinks, diet drinks, zero calorie drinks, reduced calorie drinks and foods, yogurt drinks, instant juices, instant coffee, powdered types of instant beverages, canned products, syrups, fermented soybean paste, soy sauce, vinegar, dressings, mayonnaise, ketchups, curry, soup, instant bouillon, powdered soy sauce, powdered vinegar, types of biscuits, rice biscuit, crackers, bread, chocolates, caramel, candy, chewing gum, jelly, pudding, preserved fruits and vegetables, fresh cream, jam, marmalade, flower paste, powdered milk, ice cream, sorbet, vegetables and fruits packed in bottles, canned and boiled beans, frozen beef, frozen pork, frozen goat, frozen lamb, frozen mutton, frozen poultry like frozen chicken, frozen duck and frozen turkey, frozen venison, frozen fish, frozen crustaceans like frozen crab and frozen lobster, frozen molluscs like frozen clams, frozen oysters, frozen scallops, and frozen mussels, frozen shrimps, frozen octopus, frozen squid, fresh beef, fresh pork, fresh goat, fresh lamb, fresh mutton, fresh poultry like fresh chicken, fresh duck and fresh turkey, fresh venison, fresh fish, fresh crustaceans like fresh crab and fresh lobster, fresh molluscs like fresh clams, fresh oysters, fresh scallops, and fresh mussels, fresh shrimps, fresh octopus, fresh squid, meat and foods boiled in sweetened sauce, agricultural vegetable food products, seafood, ham, sausage, fish ham, fish sausage, fish paste, deep fried fish products, dried seafood products, frozen food products, preserved seaweed, preserved meat, tobacco, medicinal products, lipsticks, and many others. In principle it can have unlimited applications.

Examples of home care products in which purified target GCGA, particularly compound A, compound B, compound C, compound D, compound E and/or compound F may be used as an antioxidant, preservative, color stabilizer, foaming suppressor, solubility enhancing agent, fragrance modifier, fragrance stabilizer, fragrance enhancer and/or deodorizer compound include, but are not limited to, laundry detergents (powder, liquid and tablet), fabric conditioners, dishwashing detergents (liquid and tablet), hard floor and surface cleaners, glass cleaners, carpet cleaners, oven cleaners, air fresheners, car shampoos, toilet care products and insect-control products and many others. In principle it can have unlimited applications.

Examples of personal care products in which purified target GCGA, particularly compound A, compound B, compound C, compound D, compound E and/or compound F may be used as an antioxidant, preservative, color stabilizer, foaming suppressor, solubility enhancing agent, fragrance modifier, fragrance stabilizer, fragrance enhancer and/or deodorizer compound include, but are not limited to, lotions, cosmetics, hair dyes, hair creams, face creams, body creams, deodorants, sunscreens, perfumes, toothpastes, bath soaps, hand soaps, shampoos, conditioners, hair masks, hair sprays, hair treatment oils, face cleansers, face masks, face moisturizers, acne creams, nail polishes, nail polish removers, shaving creams, shaving foams, shaving gels, after-shave lotions, after-shave balms, after-shave creams, after-shave fluids, shower gels, shower liquids, body sprays, anti-perspirants, skin balms, skin wipes, baby oils, baby colognes, baby shampoos, baby body shampoos, baby lotions, baby bath liquids, baby soaps, baby gel lotions, baby head-to-toe baths and baby powders and many others. In principle it can have unlimited applications.

During the manufacturing of products such as foodstuffs, drinks, pharmaceuticals, cosmetics, tabletop products, and chewing gum, the conventional methods such as mixing, kneading, dissolution, pickling, permeation, percolation, sprinkling, atomizing, infusing and other methods may be used.

Moreover, the purified target GCGA, particularly compound A, compound B, compound C, compound D, compound E and/or compound F obtained in this invention may be used in dry or liquid forms.

The purified target GCGA, particularly compound A, compound B, compound C, compound D, compound E and/or compound F can be added before or after heat treatment of food products. The amount of the purified target GCGA, particularly compound A, compound B, compound C, compound D, compound E and/or compound F depends on the purpose of usage. As discussed above, it can be added alone or in combination with other compounds.

The present invention is also directed to oxidation minimization of food, beverages, home care products and personal care products using GCGA, particularly compound A, compound B, compound C, compound D, compound E and/or compound F as an antioxidant, wherein GCGA, particularly compound A, compound B, compound C, compound D, compound E and/or compound F is present in a concentration that allows for oxidation minimization of products.

The present invention is also directed to preservation of food, beverages, home care products and personal care products using GCGA, particularly compound A, compound B, compound C, compound D, compound E and/or compound F as a preservative, wherein GCGA, particularly compound A, compound B, compound C, compound D, compound E and/or compound F is present in a concentration that allows for preservation of products.

The present invention is also directed to color stabilization of food, beverages, home care products and personal care products using GCGA, particularly compound A, compound B, compound C, compound D, compound E and/or compound F as a color stabilizer, wherein GCGA, particularly compound A, compound B, compound C, compound D, compound E and/or compound F is present in a concentration that allows for color stabilization of products.

The present invention is also directed to flavor stabilization of food, beverages, home care products and personal care products using GCGA, particularly compound A, compound B, compound C, compound D, compound E and/or compound F as a flavor stabilizer, wherein GCGA, particularly compound A, compound B, compound C, compound D, compound E and/or compound F is present in a concentration that allows for flavor stabilization of products.

The present invention is also directed to modifying a flavor profile of food, beverages, home care products and personal care products using GCGA, particularly compound A, compound B, compound C, compound D, compound E and/or compound F as a flavor with modifier properties, wherein GCGA, particularly compound A, compound B, compound C, compound D, compound E and/or compound F is present in a concentration that allows for modification of the flavor profile without imparting detectable sweetness or flavor to the product.

The present invention is also directed to foaming suppression of food, beverages, home care products and personal care products using GCGA, particularly compound A, compound B, compound C, compound D, compound E and/or compound F as a foaming suppressor, wherein GCGA, particularly compound A, compound B, compound C, compound D, compound E and/or compound F is present in a concentration that allows for foaming suppression of products.

The present invention is also directed to solubility enhancement of insoluble material in food, beverages, home care products and personal care products using GCGA, particularly compound A, compound B, compound C, compound D, compound E and/or compound F as a solubility enhancing agent, wherein GCGA, particularly compound A, compound B, compound C, compound D, compound E and/or compound F is present in a concentration that allows for solubility enhancement of products.

The present invention is also directed to fragrance modification of food, beverages, home care products and personal care products using GCGA, particularly compound A, compound B, compound C, compound D, compound E and/or compound F as a fragrance modifier, wherein GCGA, particularly compound A, compound B, compound C, compound D, compound E and/or compound F is present in a concentration that allows for fragrance modification of products.

The present invention is also directed to fragrance stabilization of food, beverages, home care products and personal care products using GCGA, particularly compound A, compound B, compound C, compound D, compound E and/or compound F as a fragrance stabilizer, wherein GCGA, particularly compound A, compound B, compound C, compound D, compound E and/or compound F is present in a concentration that allows for fragrance stabilization of products.

The present invention is also directed to fragrance enhancement of food, beverages, home care products and personal care products using GCGA, particularly compound A, compound B, compound C, compound D, compound E and/or compound F as a fragrance enhancer, wherein GCGA, particularly compound A, compound B, compound C, compound D, compound E and/or compound F is present in a concentration that allows for fragrance enhancement of products.

The present invention is also directed to deodorization of food, beverages, home care products and personal care products using GCGA, particularly compound A, compound B, compound C, compound D, compound E and/or compound F as a deodorizer, wherein GCGA, particularly compound A, compound B, compound C, compound D, compound E and/or compound F is present in a concentration that allows for deodorization of products.

As used herein, the term “color stabilizer” refers to a compound capable of stabilizing the color in a composition, such as a beverage. It is contemplated that a color stabilizer can be used alone, or in combination with other color stabilizers.

As used herein, the term “flavor stabilizer” refers to a compound capable of stabilizing the flavor in a composition, such as a beverage. It is contemplated that a flavor stabilizer can be used alone, or in combination with other flavor stabilizers.

As used herein, the terms “flavors with modifying properties” or “FMP” can be used interchangeably, and refer to ingredients that enhance, subdue or otherwise affect the taste and/or flavor profile without themselves being sweeteners or flavorings. The Flavor and Extracts Manufacturing Association (FEMA) has developed a protocol published in the November 2013 Edition of Food Technology. It is contemplated that a flavor with modifying properties (FMP) can be used alone, or in combination with other flavors.

Accordingly, the present invention also provides a method for stabilizing the color of a beverage comprising providing a beverage and adding a color stabilizer selected from GCGA, particularly compound A, wherein GCGA, particularly compound A, compound B, compound C, compound D, compound E and/or compound F are present in a concentration that allows for color stabilization.

Accordingly, the present invention also provides a method for stabilizing the flavor of a beverage comprising providing a beverage and adding a flavor stabilizer selected from GCGA, particularly compound A, wherein GCGA, particularly compound A, compound B, compound C, compound D, compound E and/or compound F are present in a concentration that allows for flavor stabilization.

Addition of GCGA, particularly compound A, compound B, compound C, compound D, compound E and/or compound F in a colored beverage may preserve the color of the beverage from about 11% to 99%, as measured by absorbance method including but not limited to the method described according to Example 8 below, such as, for example, about 11%, about 13%, about 15%, about 17%, about 19%, about 21%, about 23%, about 25%, about 27%, about 29%, about 31%, about 33%, about 35%, about 37%, about 39%, about 41%, about 43%, about 45%, about 47%, about 49%, about 51%, about 53%, about 55%, about 57%, about 59%, about 61%, about 63%, about 65%, about 67%, about 69%, about 71%, about 73%, about 75%, about 77%, about 79%, about 81%, about 83%, about 85%, about 87%, about 89%, about 91%, about 93%, about 95%, about 97%, or about 99%.

Also provided is a product comprising compound A, compound B, compound C, compound D, compound E and/or compound F, wherein the product is selected from foods, beverages, pharmaceutical compositions, tobacco products, nutraceutical compositions, oral hygiene compositions, home care products, personal care products, and cosmetic compositions.

In an embodiment, the product is selected from alcoholic beverages such as vodka, wine, beer, liquor, and sake, non-alcoholic beverages, natural juices, refreshing drinks, carbonated soft drinks, diet drinks, zero calorie drinks, reduced calorie drinks and foods, yogurt drinks, instant juices, instant coffee, powdered types of instant beverages, canned products, syrups, fermented soybean paste, soy sauce, vinegar, dressings, mayonnaise, ketchups, curry, soup, instant bouillon, powdered soy sauce, powdered vinegar, types of biscuits, rice biscuit, crackers, bread, chocolates, caramel, candy, chewing gum, jelly, pudding, preserved fruits and vegetables, fresh cream, jam, marmalade, flower paste, powdered milk, ice cream, sorbet, vegetables and fruits packed in bottles, canned and boiled beans, frozen beef, frozen pork, frozen goat, frozen lamb, frozen mutton, frozen poultry like frozen chicken, frozen duck and frozen turkey, frozen venison, frozen fish, frozen crustaceans like frozen crab and frozen lobster, frozen molluscs like frozen clams, frozen oysters, frozen scallops, and frozen mussels, frozen shrimps, frozen octopus, frozen squid, fresh beef, fresh pork, fresh goat, fresh lamb, fresh mutton, fresh poultry like fresh chicken, fresh duck and fresh turkey, fresh venison, fresh fish, fresh crustaceans like fresh crab and fresh lobster, fresh molluscs like fresh clams, fresh oysters, fresh scallops, and fresh mussels, fresh shrimps, fresh octopus, fresh squid, meat and foods boiled in sweetened sauce, agricultural vegetable food products, seafood, ham, sausage, fish ham, fish sausage, fish paste, deep fried fish products, dried seafood products, frozen food products, preserved seaweed, preserved meat, tobacco, lipsticks and medicinal products.

The product is also selected from laundry detergents (powder, liquid and tablet), fabric conditioners, dishwashing detergents (liquid and tablet), hard floor and surface cleaners, glass cleaners, carpet cleaners, oven cleaners, air fresheners, car shampoo, toilet care products, and insect-control products.

The product is furthermore selected from lotions, cosmetics, hair dyes, hair creams, face creams, body creams, deodorants, sunscreens, perfumes, toothpastes, bath soaps, hand soaps, shampoos, conditioners, hair masks, hair sprays, hair treatment oils, face cleansers, face masks, face moisturizers, acne creams, nail polishes, nail polish removers, shaving creams, shaving foams, shaving gels, after-shave lotions, after-shave balms, after-shave creams, after-shave fluids, shower gels, shower liquids, body sprays, anti-perspirants, skin balms, skin wipes, baby oils, baby colognes, baby shampoos, baby body shampoos, baby lotions, baby bath liquids, baby soaps, baby gel lotions, baby head-to-toe baths and baby powders.

In an embodiment, the product further comprises at least one additive selected from steviol glycosides, carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, caffeine, flavorants and flavoring ingredients, astringent compounds, proteins or protein hydrolysates, surfactants, emulsifiers, flavonoids, alcohols, polymers, salts thereof and combinations thereof.

In an embodiment, the product further comprises at least one functional ingredient selected from antioxidants, saponins, dietary fiber sources, fatty acids, vitamins, glucosamine, minerals, preservatives, hydration agents, probiotics, prebiotics, weight management agents, osteoporosis management agents, phytoestrogens, long chain primary aliphatic saturated alcohols, phytosterols, salts thereof, and combinations thereof.

In an embodiment, the product further comprises a compound selected from vitamin C, vitamin E, carotenoid, flavonols, flavanols, flavanones, flavones, isoflavonoids, anthocyanidins, glingerol, hydroxycinnamic acid, hydrobenzoic acid, curcumin, gallic acid, rosemary extract, rosmarinic acid, caffeic acid, carnosic acid, carnosol, butylated hydroxytoluene (BHT), butylated hydroxyanisole (BHA), tert-butylated hydroxyquinone (TBHQ), propyl 3,4,5-trihydroxybenzoate (PG), and 1,2,3-trihydroxybenzene (Pyrogallol-PY), salts thereof, and combinations thereof.

Also provided is a method for increasing the stability of a product towards oxidation, comprising the steps of adding a compound selected from compound A, compound B, compound C, compound D, compound E, compound F, and combinations thereof to a product selected from beverage products, food products, and home care products, wherein the compound is present in an amount ranging from about 0.005% to about 0.5% by weight, to generate a product having improved stability towards oxidation throughout storage compared to a control product that does not contain the compound. In an embodiment, the compound is present in an amount ranging from about 0.005% to about 0.5% by weight, such as, for example, about 0.005% by weight, about 0.01% by weight, about 0.02% by weight, about 0.03% by weight, about 0.04% by weight, about 0.05% by weight, about 0.06% by weight, about 0.07% by weight, about 0.08% by weight, about 0.09% by weight, about 0.10% by weight, about 0.11 by weight, about 0.12% by weight, about 0.13% by weight, about 0.14% by weight, about 0.15% by weight, about 0.16% by weight, about 0.17% by weight, about 0.18% by weight, about 0.19% by weight, about 0.20% by weight, about 0.21% by weight, about 0.22% by weight, about 0.23% by weight, about 0.24% by weight, about 0.25% by weight, about 0.26% by weight, about 0.27% by weight, about 0.28% by weight, about 0.29% by weight, about 0.30% by weight, about 0.31% by weight, about 0.32% by weight, about 0.33% by weight, about 0.34% by weight, about 0.35% by weight, about 0.36% by weight, about 0.37% by weight, about 0.38% by weight, about 0.39% by weight, about 0.40% by weight, about 0.41% by weight, about 0.42% by weight, about 0.43% by weight, about 0.44% by weight, about 0.45% by weight, about 0.46% by weight, about 0.47% by weight, about 0.48% by weight, about 0.49% by weight or about 0.5% by weight.

Also provided is a method for preserving a beverage product, a food product, a home care product or a personal care product, comprising providing a beverage product, a food product, a home care product or a personal care product; and adding a composition comprising compound A, compound B, compound C, compound D, compound E and compound F, wherein compound A, compound B, compound C, compound D, compound E and compound F are present in a concentration at about 0.005% to about 0.5% by weight. In an embodiment, the compound is present in an amount ranging from about 0.005% to about 0.5% by weight, such as, for example, about 0.005% by weight, about 0.01% by weight, about 0.02% by weight, about 0.03% by weight, about 0.04% by weight, about 0.05% by weight, about 0.06% by weight, about 0.07% by weight, about 0.08% by weight, about 0.09% by weight, about 0.10% by weight, about 0.11 by weight, about 0.12% by weight, about 0.13% by weight, about 0.14% by weight, about 0.15% by weight, about 0.16% by weight, about 0.17% by weight, about 0.18% by weight, about 0.19% by weight, about 0.20% by weight, about 0.21% by weight, about 0.22% by weight, about 0.23% by weight, about 0.24% by weight, about 0.25% by weight, about 0.26% by weight, about 0.27% by weight, about 0.28% by weight, about 0.29% by weight, about 0.30% by weight, about 0.31% by weight, about 0.32% by weight, about 0.33% by weight, about 0.34% by weight, about 0.35% by weight, about 0.36% by weight, about 0.37% by weight, about 0.38% by weight, about 0.39% by weight, about 0.40% by weight, about 0.41% by weight, about 0.42% by weight, about 0.43% by weight, about 0.44% by weight, about 0.45% by weight, about 0.46% by weight, about 0.47% by weight, about 0.48% by weight, about 0.49% by weight or about 0.5% by weight.

This invention also provides a method for stabilizing the color of a beverage product, a food product, a home care product or a personal care product, comprising the steps of adding a compound selected from compound A, compound B, compound C, compound D, compound E, compound F, and combinations thereof to a product selected from beverage products, food products, and home care products, wherein the compound is present in an amount ranging from about 0.005% to about 0.5% by weight, to generate a product having improved color stability compared to a control product that does not contain the compound. In an embodiment, the compound is present in an amount ranging from about 0.005% to about 0.5% by weight, such as, for example, about 0.005% by weight, about 0.01% by weight, about 0.02% by weight, about 0.03% by weight, about 0.04% by weight, about 0.05% by weight, about 0.06% by weight, about 0.07% by weight, about 0.08% by weight, about 0.09% by weight, about 0.10% by weight, about 0.11 by weight, about 0.12% by weight, about 0.13% by weight, about 0.14% by weight, about 0.15% by weight, about 0.16% by weight, about 0.17% by weight, about 0.18% by weight, about 0.19% by weight, about 0.20% by weight, about 0.21% by weight, about 0.22% by weight, about 0.23% by weight, about 0.24% by weight, about 0.25% by weight, about 0.26% by weight, about 0.27% by weight, about 0.28% by weight, about 0.29% by weight, about 0.30% by weight, about 0.31% by weight, about 0.32% by weight, about 0.33% by weight, about 0.34% by weight, about 0.35% by weight, about 0.36% by weight, about 0.37% by weight, about 0.38% by weight, about 0.39% by weight, about 0.40% by weight, about 0.41% by weight, about 0.42% by weight, about 0.43% by weight, about 0.44% by weight, about 0.45% by weight, about 0.46% by weight, about 0.47% by weight, about 0.48% by weight, about 0.49% by weight or about 0.5% by weight.

This invention also provides a method for stabilizing the flavor of a beverage product or a food product, comprising the steps of adding a compound selected from compound A, compound B, compound C, compound D, compound E, compound F, and combinations thereof to a product selected from beverage products, food products, and home care products, wherein the compound is present in an amount ranging from about 0.005% to about 0.5% by weight, to generate a product having improved flavor stability compared to a control product that does not contain the compound. In an embodiment, the compound is present in an amount ranging from about 0.005% to about 0.5% by weight, such as, for example, about 0.005% by weight, about 0.01% by weight, about 0.02% by weight, about 0.03% by weight, about 0.04% by weight, about 0.05% by weight, about 0.06% by weight, about 0.07% by weight, about 0.08% by weight, about 0.09% by weight, about 0.10% by weight, about 0.11 by weight, about 0.12% by weight, about 0.13% by weight, about 0.14% by weight, about 0.15% by weight, about 0.16% by weight, about 0.17% by weight, about 0.18% by weight, about 0.19% by weight, about 0.20% by weight, about 0.21% by weight, about 0.22% by weight, about 0.23% by weight, about 0.24% by weight, about 0.25% by weight, about 0.26% by weight, about 0.27% by weight, about 0.28% by weight, about 0.29% by weight, about 0.30% by weight, about 0.31% by weight, about 0.32% by weight, about 0.33% by weight, about 0.34% by weight, about 0.35% by weight, about 0.36% by weight, about 0.37% by weight, about 0.38% by weight, about 0.39% by weight, about 0.40% by weight, about 0.41% by weight, about 0.42% by weight, about 0.43% by weight, about 0.44% by weight, about 0.45% by weight, about 0.46% by weight, about 0.47% by weight, about 0.48% by weight, about 0.49% by weight or about 0.5% by weight.

This invention also provides a method for modifying a flavor profile of a beverage product or a food product, comprising providing a beverage product or a food product; and adding a composition comprising compound A, compound B, compound C, compound D, compound E and compound F, wherein compound A, compound B, compound C, compound D, compound E and compound F of are present in a concentration at about 0.005% to about 0.5% by weight, such as, for example, about 0.005% by weight, about 0.01% by weight, about 0.02% by weight, about 0.03% by weight, about 0.04% by weight, about 0.05% by weight, about 0.06% by weight, about 0.07% by weight, about 0.08% by weight, about 0.09% by weight, about 0.10% by weight, about 0.11 by weight, about 0.12% by weight, about 0.13% by weight, about 0.14% by weight, about 0.15% by weight, about 0.16% by weight, about 0.17% by weight, about 0.18% by weight, about 0.19% by weight, about 0.20% by weight, about 0.21% by weight, about 0.22% by weight, about 0.23% by weight, about 0.24% by weight, about 0.25% by weight, about 0.26% by weight, about 0.27% by weight, about 0.28% by weight, about 0.29% by weight, about 0.30% by weight, about 0.31% by weight, about 0.32% by weight, about 0.33% by weight, about 0.34% by weight, about 0.35% by weight, about 0.36% by weight, about 0.37% by weight, about 0.38% by weight, about 0.39% by weight, about 0.40% by weight, about 0.41% by weight, about 0.42% by weight, about 0.43% by weight, about 0.44% by weight, about 0.45% by weight, about 0.46% by weight, about 0.47% by weight, about 0.48% by weight, about 0.49% by weight or about 0.5% by weight.

This invention also provides a method for suppressing foaming of a beverage product, a food product, a home care product or a personal care product, comprising the steps of adding a compound selected from compound A, compound B, compound C, compound D, compound E, compound F, and combinations thereof to a product selected from beverage products, food products, and home care products, wherein the compound is present in an amount ranging from about 0.005% to about 0.5% by weight, to generate a product with suppressed foaming. In an embodiment, the compound is present in an amount ranging from about 0.005% to about 0.5% by weight, such as, for example, about 0.005% by weight, about 0.01% by weight, about 0.02% by weight, about 0.03% by weight, about 0.04% by weight, about 0.05% by weight, about 0.06% by weight, about 0.07% by weight, about 0.08% by weight, about 0.09% by weight, about 0.10% by weight, about 0.11 by weight, about 0.12% by weight, about 0.13% by weight, about 0.14% by weight, about 0.15% by weight, about 0.16% by weight, about 0.17% by weight, about 0.18% by weight, about 0.19% by weight, about 0.20% by weight, about 0.21% by weight, about 0.22% by weight, about 0.23% by weight, about 0.24% by weight, about 0.25% by weight, about 0.26% by weight, about 0.27% by weight, about 0.28% by weight, about 0.29% by weight, about 0.30% by weight, about 0.31% by weight, about 0.32% by weight, about 0.33% by weight, about 0.34% by weight, about 0.35% by weight, about 0.36% by weight, about 0.37% by weight, about 0.38% by weight, about 0.39% by weight, about 0.40% by weight, about 0.41% by weight, about 0.42% by weight, about 0.43% by weight, about 0.44% by weight, about 0.45% by weight, about 0.46% by weight, about 0.47% by weight, about 0.48% by weight, about 0.49% by weight or about 0.5% by weight.

This invention also provides a method for enhancing the solubility of insoluble material in a beverage product, a food product, a home care product or a personal care product, comprising the steps of adding a compound selected from compound A, compound B, compound C, compound D, compound E, compound F, and combinations thereof to a product selected from beverage products, food products, and home care products, wherein the compound is present in an amount ranging from about 0.005% to about 0.5% by weight, to generate a product having less insoluble material compared to a control product that does not contain the compound. In an embodiment, the compound is present in an amount ranging from about 0.005% to about 0.5% by weight, such as, for example, about 0.005% by weight, about 0.01% by weight, about 0.02% by weight, about 0.03% by weight, about 0.04% by weight, about 0.05% by weight, about 0.06% by weight, about 0.07% by weight, about 0.08% by weight, about 0.09% by weight, about 0.10% by weight, about 0.11 by weight, about 0.12% by weight, about 0.13% by weight, about 0.14% by weight, about 0.15% by weight, about 0.16% by weight, about 0.17% by weight, about 0.18% by weight, about 0.19% by weight, about 0.20% by weight, about 0.21% by weight, about 0.22% by weight, about 0.23% by weight, about 0.24% by weight, about 0.25% by weight, about 0.26% by weight, about 0.27% by weight, about 0.28% by weight, about 0.29% by weight, about 0.30% by weight, about 0.31% by weight, about 0.32% by weight, about 0.33% by weight, about 0.34% by weight, about 0.35% by weight, about 0.36% by weight, about 0.37% by weight, about 0.38% by weight, about 0.39% by weight, about 0.40% by weight, about 0.41% by weight, about 0.42% by weight, about 0.43% by weight, about 0.44% by weight, about 0.45% by weight, about 0.46% by weight, about 0.47% by weight, about 0.48% by weight, about 0.49% by weight or about 0.5% by weight.

This invention also provides a method for modifying the fragrance of a beverage product, a food product, a home care product or a personal care product, comprising the steps of adding a compound selected from compound A, compound B, compound C, compound D, compound E, compound F, and combinations thereof to a product selected from beverage products, food products, and home care products, wherein the compound is present in an amount ranging from about 0.005% to about 0.5% by weight, to generate a product having a modified fragrance compared to a control product that does not contain the compound. In an embodiment, the compound is present in an amount ranging from about 0.005% to about 0.5% by weight, such as, for example, about 0.005% by weight, about 0.01% by weight, about 0.02% by weight, about 0.03% by weight, about 0.04% by weight, about 0.05% by weight, about 0.06% by weight, about 0.07% by weight, about 0.08% by weight, about 0.09% by weight, about 0.10% by weight, about 0.11 by weight, about 0.12% by weight, about 0.13% by weight, about 0.14% by weight, about 0.15% by weight, about 0.16% by weight, about 0.17% by weight, about 0.18% by weight, about 0.19% by weight, about 0.20% by weight, about 0.21% by weight, about 0.22% by weight, about 0.23% by weight, about 0.24% by weight, about 0.25% by weight, about 0.26% by weight, about 0.27% by weight, about 0.28% by weight, about 0.29% by weight, about 0.30% by weight, about 0.31% by weight, about 0.32% by weight, about 0.33% by weight, about 0.34% by weight, about 0.35% by weight, about 0.36% by weight, about 0.37% by weight, about 0.38% by weight, about 0.39% by weight, about 0.40% by weight, about 0.41% by weight, about 0.42% by weight, about 0.43% by weight, about 0.44% by weight, about 0.45% by weight, about 0.46% by weight, about 0.47% by weight, about 0.48% by weight, about 0.49% by weight or about 0.5% by weight.

This invention also provides a method for stabilizing the fragrance of a beverage product, a food product, a home care product or a personal care product, comprising the steps of adding a compound selected from compound A, compound B, compound C, compound D, compound E, compound F, and combinations thereof to a product selected from beverage products, food products, and home care products, wherein the compound is present in an amount ranging from about 0.005% to about 0.5% by weight, to generate a product having enhanced fragrance stability compared to a control product that does not contain the compound. In an embodiment, the compound is present in an amount ranging from about 0.005% to about 0.5% by weight, such as, for example, about 0.005% by weight, about 0.01% by weight, about 0.02% by weight, about 0.03% by weight, about 0.04% by weight, about 0.05% by weight, about 0.06% by weight, about 0.07% by weight, about 0.08% by weight, about 0.09% by weight, about 0.10% by weight, about 0.11 by weight, about 0.12% by weight, about 0.13% by weight, about 0.14% by weight, about 0.15% by weight, about 0.16% by weight, about 0.17% by weight, about 0.18% by weight, about 0.19% by weight, about 0.20% by weight, about 0.21% by weight, about 0.22% by weight, about 0.23% by weight, about 0.24% by weight, about 0.25% by weight, about 0.26% by weight, about 0.27% by weight, about 0.28% by weight, about 0.29% by weight, about 0.30% by weight, about 0.31% by weight, about 0.32% by weight, about 0.33% by weight, about 0.34% by weight, about 0.35% by weight, about 0.36% by weight, about 0.37% by weight, about 0.38% by weight, about 0.39% by weight, about 0.40% by weight, about 0.41% by weight, about 0.42% by weight, about 0.43% by weight, about 0.44% by weight, about 0.45% by weight, about 0.46% by weight, about 0.47% by weight, about 0.48% by weight, about 0.49% by weight or about 0.5% by weight.

This invention also provides a method for enhancing the fragrance of a beverage product, a food product, a home care product or a personal care product, comprising the steps of adding a compound selected from compound A, compound B, compound C, compound D, compound E, compound F, and combinations thereof to a product selected from beverage products, food products, and home care products, wherein the compound is present in an amount ranging from about 0.005% to about 0.5% by weight, to generate a product having enhanced fragrance compared to a control product that does not contain the compound. In an embodiment, the compound is present in an amount ranging from about 0.005% to about 0.5% by weight, such as, for example, about 0.005% by weight, about 0.01% by weight, about 0.02% by weight, about 0.03% by weight, about 0.04% by weight, about 0.05% by weight, about 0.06% by weight, about 0.07% by weight, about 0.08% by weight, about 0.09% by weight, about 0.10% by weight, about 0.11 by weight, about 0.12% by weight, about 0.13% by weight, about 0.14% by weight, about 0.15% by weight, about 0.16% by weight, about 0.17% by weight, about 0.18% by weight, about 0.19% by weight, about 0.20% by weight, about 0.21% by weight, about 0.22% by weight, about 0.23% by weight, about 0.24% by weight, about 0.25% by weight, about 0.26% by weight, about 0.27% by weight, about 0.28% by weight, about 0.29% by weight, about 0.30% by weight, about 0.31% by weight, about 0.32% by weight, about 0.33% by weight, about 0.34% by weight, about 0.35% by weight, about 0.36% by weight, about 0.37% by weight, about 0.38% by weight, about 0.39% by weight, about 0.40% by weight, about 0.41% by weight, about 0.42% by weight, about 0.43% by weight, about 0.44% by weight, about 0.45% by weight, about 0.46% by weight, about 0.47% by weight, about 0.48% by weight, about 0.49% by weight or about 0.5% by weight.

This invention also provides a method for deodorizing a beverage product, a food product, a home care product or a personal care product, comprising the steps of adding a compound selected from compound A, compound B, compound C, compound D, compound E, compound F, and combinations thereof to a product selected from beverage products, food products, and home care products, wherein the compound is present in an amount ranging from about 0.005% to about 0.5% by weight, to generate a product with less or no odor compared to a control product that does not contain the compound. In an embodiment, the compound is present in an amount ranging from about 0.005% to about 0.5% by weight, such as, for example, about 0.005% by weight, about 0.01% by weight, about 0.02% by weight, about 0.03% by weight, about 0.04% by weight, about 0.05% by weight, about 0.06% by weight, about 0.07% by weight, about 0.08% by weight, about 0.09% by weight, about 0.10% by weight, about 0.11 by weight, about 0.12% by weight, about 0.13% by weight, about 0.14% by weight, about 0.15% by weight, about 0.16% by weight, about 0.17% by weight, about 0.18% by weight, about 0.19% by weight, about 0.20% by weight, about 0.21% by weight, about 0.22% by weight, about 0.23% by weight, about 0.24% by weight, about 0.25% by weight, about 0.26% by weight, about 0.27% by weight, about 0.28% by weight, about 0.29% by weight, about 0.30% by weight, about 0.31% by weight, about 0.32% by weight, about 0.33% by weight, about 0.34% by weight, about 0.35% by weight, about 0.36% by weight, about 0.37% by weight, about 0.38% by weight, about 0.39% by weight, about 0.40% by weight, about 0.41% by weight, about 0.42% by weight, about 0.43% by weight, about 0.44% by weight, about 0.45% by weight, about 0.46% by weight, about 0.47% by weight, about 0.48% by weight, about 0.49% by weight or about 0.5% by weight.

The following examples illustrate preferred embodiments of the invention for the preparation of purified target GCGA, particularly compound A, compound B, compound C, compound D, compound E and compound F. It will be understood that the invention is not limited to the materials, proportions, conditions and procedures set forth in the examples, which are only illustrative.

### EXAMPLES

### Example 1

### Synthesis of GCGA Composition

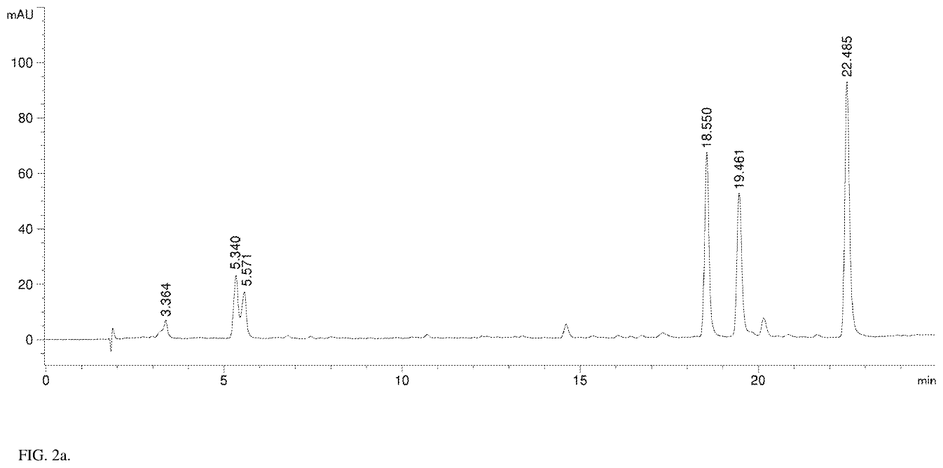

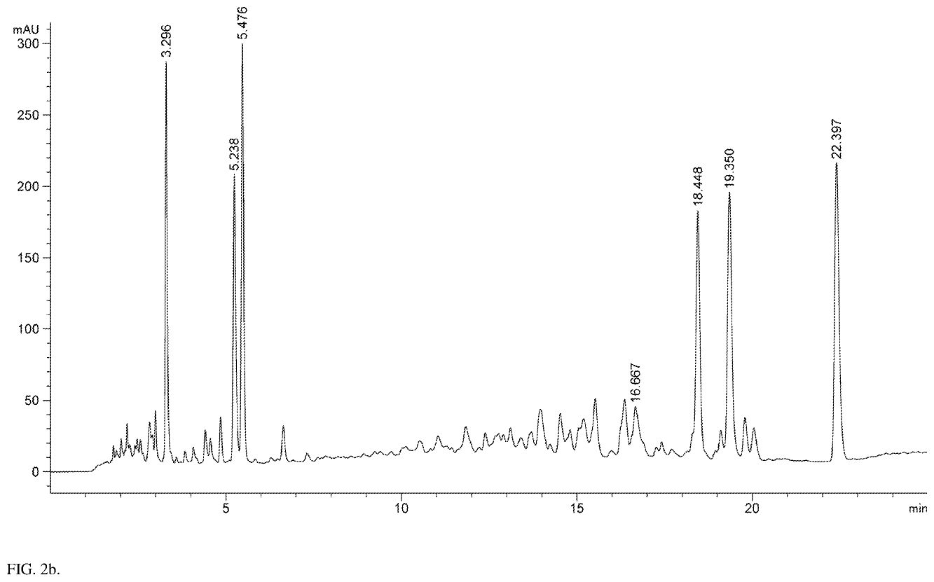

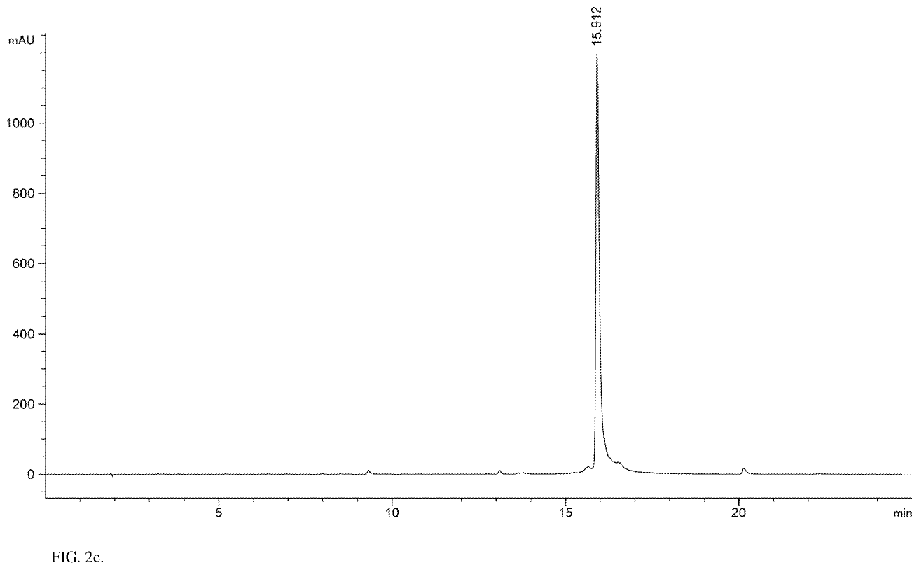

GCGA were synthesized directly from a Stevia CGA composition (see FIG. 2a) utilizing the enzyme CGTase. One KNU (Kilo Novo Units) of CGTase was defined as the amount of enzyme that breaks down 5.26 g starch per hour.

First, 1375 mL of 0.1% sodium bicarbonate solution were mixed with 51.5 g of Stevia CGA composition, 257.5 g maltodextrin DE10-15 for 30 mins at 40° C.

After dissolving the components, 145.5 KNU of CGTase were added to make a reaction mixture containing approximately 105.8 KNU/L CGTase, 37.5 g/L CGA composition, 187.4 g/L maltodextrin DE10-15 and 0.1% sodium bicarbonate solution.

The reaction mixture was agitated at 40° C. for 7 days. The content of compound A and various GCGA at the end of the reaction (7 days) was analyzed by HPLC as described in EXAMPLE 2.

### Example 2

### HPLC Analysis

Reaction mixture samples were filtered using cardboard with pore size of 3 μm, the filtrates were diluted 5 times and used as samples for HPLC analysis. HPLC assay was carried out on Agilent HP 1200 HPLC system, comprised of a pump, a column thermostat, an auto sampler, a UV detector capable of background correction and a data acquisition system. Analytes were separated using Agilent Poroshell 120 SB-C18, 4.6 mm×150 mm, 2.7 μm at 40° C. The mobile phase consisted of:
0.1% (v/v) formic acid, and
acetonitrile.

Gradient elution was set based on Table 1.

TABLE 1
Gradient elution of HPLC assay for CGA and GCGA

| Run Time (min) | 0.1% (v/v) Formic acid, % | Acetonitrile, % |
|---|---|---|
| 0 | 90 | 10 |
| 20 | 80 | 20 |
| 25 | 70 | 30 |
| 35 | 60 | 40 |
| 40 | 60 | 40 |

Total run time was 40 minutes. The column temperature was maintained at 40° C. The injection volume was 10 μL. CGA and GCGA species were detected by UV at 324 nm.

Table 2 shows for each time point the conversion of CGA into identified GCGA species (area percentage). The chromatograms of the starting material CGA and the reaction mixture at 7 days are shown in FIGS. 2a and 2b, respectively. Those skilled in the art will appreciate that retention times (rt) can occasionally vary with changes in solvent and/or equipment.

TABLE 2
Synthesis of compound A (GCGA rt 16.667)
and various GCGA from CGA.

| | peak area (%) | |
|---|---|---|
| | reaction | reaction |
| Peak name | time 0 hr | time 7 days |
| GCGA rt 1.882 | 0 | 0.07 |
| GCGA rt 2.015 | 0 | 0.24 |
| GCGA rt 2.121 | 0 | 0.06 |
| GCGA rt 2.180 | 0 | 0.49 |
| GCGA rt 2.256 | 0 | 0.20 |
| GCGA rt 2.467 | 0 | 0.51 |
| GCGA rt 2.566 | 0 | 0.33 |
| GCGA rt 2.639 | 0 | 0.10 |
| GCGA rt 2.829 | 0 | 1.09 |
| GCGA rt 2.902 | 0 | 0.46 |
| GCGA rt 2.994 | 0 | 0.93 |
| 5-O-caffeoyl quinic acid rt 3.296 | 2.86 | 8.01 |
| GCGA rt 3.839 | 0 | 0.23 |
| GCGA rt 4.075 | 0 | 0.37 |
| GCGA rt 4.411 | 0 | 0.79 |
| GCGA rt 4.560 | 0 | 0.52 |
| GCGA rt 4.848 | 0 | 1.01 |
| 3-O-caffeoyl quinic acid rt 5.238 | 8.53 | 8.16 |
| 4-O-caffeoyl quinic acid rt 5.476 | 6.52 | 10.73 |
| GCGA rt 9.243 | 0 | 0.19 |
| GCGA rt 9.398 | 0 | 0.14 |
| GCGA rt 10.029 | 0 | 0.17 |
| GCGA rt 10.133 | 0 | 0.21 |
| GCGA rt 10.834 | 0 | 0.09 |
| GCGA rt 11.047 | 0 | 0.96 |
| GCGA rt 11.834 | 0 | 1.40 |
| GCGA rt 12.391 | 0 | 0.60 |
| GCGA rt 12.599 | 0 | 0.15 |
| GCGA rt 12.763 | 0 | 0.93 |
| GCGA rt 12.905 | 0 | 0.48 |
| GCGA rt 13.105 | 0 | 0.77 |
| GCGA rt 13.647 | 0 | 0.70 |
| GCGA rt 13.701 | 0 | 0.67 |
| GCGA rt 13.967 | 0 | 2.54 |
| GCGA rt 14.803 | 0 | 1.25 |
| GCGA rt 15.066 | 0 | 1.03 |
| GCGA rt 15.192 | 0 | 2.02 |
| GCGA rt 15.522 | 0 | 3.18 |
| GCGA rt 16.296 | 0 | 0.96 |
| GCGA rt 16.357 | 0 | 2.34 |
| GCGA rt 16.667 | 0 | 3.68 |
| GCGA rt 17.412 | 0 | 0.52 |
| GCGA rt 18.321 | 0 | 0.48 |
| 3,4-di-O-caffeoyl quinic acid rt 18.448 | 24.33 | 10.52 |
| GCGA rt 19.102 | 0 | 1.07 |
| 3,5-di-O-caffeoyl quinic acid rt 19.350 | 20.78 | 12.48 |
| GCGA rt 19.790 | 0 | 1.69 |
| 4,5-di-O-caffeoyl quinic acid 22.397 | 36.98 | 14.47 |

### Example 3

### Purification of Compound A

The reaction mixture of EXAMPLE 1 (after 7 days), was filtered. The filtrate was loaded into a column containing 1800 mL YWD03 (Cangzhou Yuanwei, China) resin pre-equilibrated with water. The resin was washed with 5 bed volumes of water and the water effluent from this step was discarded.

GCGA were eluted from the YWD03 resin column with 5 bed volumes of 30% v/v ethanol/water. The effluent from this step was collected and dried under vacuum at 40° C. to yield 39 g of dried solid product.

The obtained dried solid was mixed with 91% v/v acetone/water in the ratio of 1:20 (solid:solvent). The insoluble material was subjected to centrifugation, whereby the supernatant was removed, and the centrifugation pellet was dried at 40° C. under vacuum and processed further as described below.

The dried pellet was dissolved in water and subjected to further fractionation and separation by two HPLC steps, using the conditions listed in Table 3a and Table 3b below. GCGA fractions containing purified compound A from multiple HPLC separation runs were combined and freeze-dried.

TABLE 3a
Conditions for HPLC (Step 1)

| Column | Zorbax SB-C18, 9.4 mm × 50 mm, 5 μm | | |
|---|---|---|---|
| Temperature | 40° C. | | |
| | Gradient - 0.1% (v/v) formic acid, Acetonitrile | | |
| | | 0.1% Formic | |
| | Run Time (min) | acid, % | Acetonitrile, % |
| Mobile Phase | 0 | 90 | 10 |
| | 2.0 | 80 | 20 |
| | 2.5 | 70 | 30 |
| | 3.5 | 60 | 40 |
| | 4.0 | 60 | 40 |

| | |
|---|---|
| Flow rate | 10.0 mL/min |
| Injection | 200 μL |
| Stop time | 4 mins |
| Autosampler | Ambient |
| temperature | |
| Detection | UV at 324 nm |

TABLE 3b
Conditions for HPLC (Step 2)

| Column | Agilent Poroshell 120 SB- C18, 4.6 mm × 150 mm, | | |
|---|---|---|---|
| | 2.7 μm | | |
| Temperature | 40° C. | | |
| | Gradient - 0.1% (v/v) formic acid, Acetonitrile | | |
| | | 0.1% (v/v) | |
| | Run Time (min) | Formic acid, % | Acetonitrile, % |
| Mobile Phase | 0 | 90 | 10 |
| | 35 | 80 | 20 |

| | |
|---|---|
| Flow rate | 1.0 mL/min |
| Injection | 10 μL |
| Stop time | 35 mins |
| Autosampler | Ambient |
| temperature | |
| Detection | UV at 324 nm |

The purity of obtained fraction was evaluated by LCMS method described in EXAMPLE 4 below. The chromatogram of purified compound A is shown in FIG. 2c.

### Example 4

### Structure Elucidation of Compound A

Compound A obtained in EXAMPLE 3 was dissolved in CD₃OD and subjected to multiple NMR analyses. 1H NMR referenced to 3.31 ppm of the residual 1H signal of solvent CD₃OD. A large water peak was seen at 4.87 ppm. The 1H spectrum showed overall 24 signals, listed in Table 4a in numerical order, along with the multiplicity, integration, coupling constants, and selected correlations.

TABLE 4a
Peak list of 1H NMR, with COSY and selected ROESY correlations. Ax and eq refer to
axial and equatorial designations for the quinic acid moiety; Glc refers to glucose.
One caffeoyl unit is denoted with prime (′), another with double-prime (″).

| | | | | | | | | selected | selected |
|---|---|---|---|---|---|---|---|---|---|
| | 1H | | J | J | J | | | COSY | ROESY |
| Entry | ppm | type | Hz | Hz | Hz | Int | Assignment | correlations | correlations |
| 1 | 7.62 | d | 15.9 | | | 1H | 7′ | 8′, 2′, 6′ | |
| 2 | 7.51 | d | 15.9 | | | 1H | 7″ | 8″, 2″, 6″ | |
| 3 | 7.26 | d | 8.4 | | | 1H | 5′ | 6′ | |
| 4 | 7.09 | d | 2.1 | | | 1H | 2′ | 7′, 6′ | |
| 5 | 7.01 | dd | 8.4 | 2.1 | | 1H | 6′ | 7′, 2′, 5′ | |
| 6 | 7.00 | d | 2.0 | | | 1H | 2″ | 7″, 6″ | |
| 7 | 6.90 | dd | 8.2 | 2.1 | | 1H | 6″ | 7″, 2″, 5″ | |
| 8 | 6.74 | d | 8.2 | | | 1H | 5″ | 6″ | |
| 9 | 6.38 | d | 15.9 | | | 1H | 8′ | 7′ | |
| 10 | 6.19 | d | 15.9 | | | 1H | 8″ | 7″ | |
| 11 | 5.63 | ddd | 9.3 | 9.3 | 4.7 | 1H | H5ax | H4ax | |
| | | | | | | | | H6eq, | |
| | | | | | | | | H6ax | |
| 12 | 5.42 | d | 3.7 | | | 1H | H1-Glc | H2-Glc | 5′ |
| 13 | 5.12 | dd | 9.0 | 3.1 | | 1H | H4ax | H5ax, | H2ax, |
| | | | | | | | | H3eq | H6ax |
| 14 | 4.38 | ddd | 4.4 | 3.3 | 3.2 | 1H | H3eq | H4ax, | |
| | | | | | | | | H2ax, | |
| | | | | | | | | H2eq | |
| 15 | 3.87 | dd | 9.3 | 9.3 | | 1H | H3-Glc | H2-Glc, | H5-Glc |
| | | | | | | | | H4-Glc | |
| 16 | 3.77 | dd | 12.0 | 2.3 | | 1H | H6a-Glc | H5-Glc, | |
| | | | | | | | | H6b-Glc | |
| 17 | 3.70 | dd | 12.0 | 5.2 | | 1H | H6b-Glc | H5-Glc, | |
| | | | | | | | | H6a-Glc | |
| 18 | 3.66 | ddd | 10.1 | 5.1 | 2.3 | 1H | H5-Glc | H4-Glc, | |
| | | | | | | | | H6a-Glc, | |
| | | | | | | | | H6b-Glc | |
| 19 | 3.59 | dd | 9.8 | 3.7 | | 1H | H2-Glc | H1-Glc, | H4-Glc |
| | | | | | | | | H3-Glc | |
| 20 | 3.42 | dd | 9.5 | 9.5 | | 1H | H4-Glc | H3-Glc, | |
| | | | | | | | | H5-Glc | |
| 21 | 2.32 | br dd | 14.3 | 3.3 | | 1H | H2ax | H3eq, | |
| | | | | | | | | H2eq | |
| 22 | 2.28 | m | | | | 1H | H6eq | H5ax, | |
| | | | | | | | | H6ax | |
| 23 | 2.24 | dd | 13.4 | 9.8 | | 1H | H6ax | H5ax, | |
| | | | | | | | | H6eq | |
| 24 | 2.12 | br dd | 14.1 | 3.8 | | 1H | H2eq | H3eq, | |
| | | | | | | | | H2ax | |

In the 13C NMR spectrum. 31 signals were seen and summarized in Table 4b. The solvent signal was referenced at 49.00 ppm. Twenty-one of these signals showed correlations to 1H by use of HSQC, where three are CH2, and eighteen are CH. The remaining ten 13C signals without HSQC correlations were assigned based on HMBC correlations.

TABLE 4b
13C NMR peak list, along with HSQC correlations and selected HMBC
correlations. Ax and eq refer to axial and equatorial designations
for the quinic acid moiety; Glc refers to glucose. One caffeoyl
unit is denoted with prime (′), another with double-prime (″).

| | | | | HSQC | Selected |
|---|---|---|---|---|---|
| | | | | correlations, | HMBC |
| Entry | 13C ppm | Type | Assignment | 1H ppm | correlations |
| 1 | 176.8 | COOH | C7 | | H2ax, H2eq, |
| | | | | | H6ax, H6eq |
| 2 | 168.22 | COOR | C9″ | | H5ax, H8″ |
| 3 | 168.15 | COOR | C9′ | | H4ax, H8′ |
| 4 | 149.7 | C | C4″ | | H2″, H6″ |
| 5 | 148.9 | C | C3′ | | H2′, H5′, |
| | | | | | H7′ |
| 6 | 148.6 | C | C4′ | | H1-Glc, H6′, |
| | | | | | H2′ |
| 7 | 147.6 | CH | C7″ | 7.51 | |
| 8 | 146.9 | CH | C7′ | 7.62 | |
| 9 | 146.8 | C | C3″ | | H2″, H5″, |
| | | | | | H7″ (partial) |
| 10 | 131.0 | C | C1′ | | H8′ |
| 11 | 127.6 | C | C1″ | | H8″ |
| 12 | 123.1 | CH | C6″ | 6.90 | |
| 13 | 122.3 | CH | C6′ | 7.01 | |
| 14 | 118.4 | CH | C5′ | 7.26 | |
| 15 | 116.9 | CH | C8′ | 6.38 | |
| 16 | 116.5 | CH | C5″ | 6.74 | |
| 17 | 116.1 | CH | C2′ | 7.09 | |
| 18 | 115.2 | CH | C2″ | 7.00 | |
| 19 | 114.7 | CH | C8″ | 6.19 | |
| 20 | 100.8 | CH | C1-Glc | 5.42 | |
| 21 | 76.0 | C | C1 | | H2eq, H6eq |
| 22 | 75.9 | CH | C4 | 5.12 | |
| 23 | 74.8 | CH | C3-Glc | 3.87 | |
| 24 | 74.7 | CH | C5-Glc | 3.66 | |
| 25 | 73.4 | CH | C2-Glc | 3.59 | |
| 26 | 71.3 | CH | C4-Glc | 3.42 | |
| 27 | 69.3 | CH | C3 | 4.38 | |
| 28 | 69.0 | CH | C5 | 5.63 | |
| 29 | 62.3 | CH2 | C6-Glc | 3.77, 3.70 | |
| 30 | 39.3 | CH2 | C6 | 2.28, 2.24 | |
| 31 | 38.4 | CH2 | C2 | 2.32, 2.12 | |

Interpretation of 1H NMR aided by TOCSY revealed six separate 1H spin systems: two trans-disubstituted alkene, two 1,3,4-trisubstituted aromatic rings, one glucose, and a quinic acid.

The two alkenes were assigned trans geometry due to the large 3J coupling constant 15.9 Hz seen for the protons at 7.62, 7.51, 6.38 and 6.19 ppm. The two aromatic rings are both assigned a 1,3,4-trisubstitution pattern, i.e. with protons at positions 2, 5 and 6. This is supported by the observation of two signals at 7.26 and 6.74 ppm (d, 3J˜8.3 Hz) assigned to H5′ and H5″, two signals at 7.01 and 6.90 ppm (dd, 3J˜8.3 Hz, 4J˜2.1 Hz) assigned to H6′ and H6″, and two signals at 7.09 and 7.00 ppm (d, 4J˜2.1 Hz) assigned to H2′ and H2″.

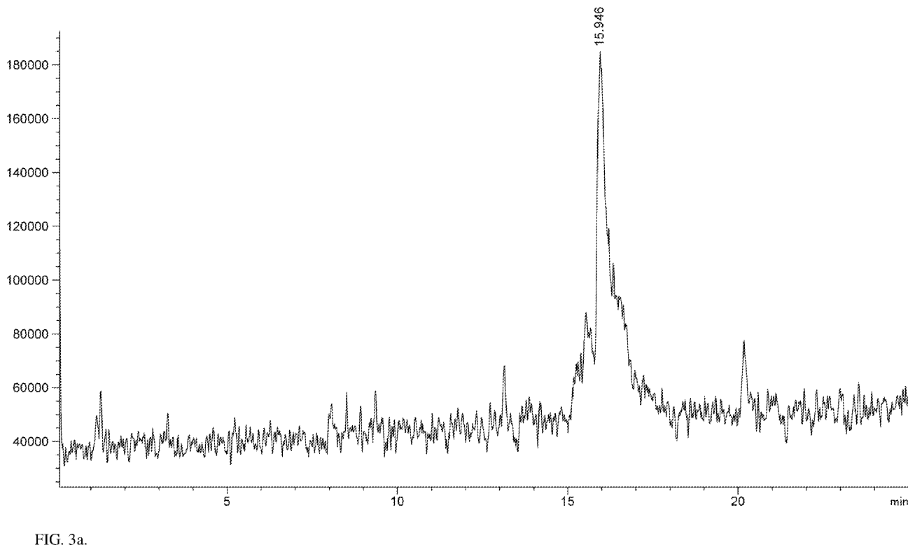

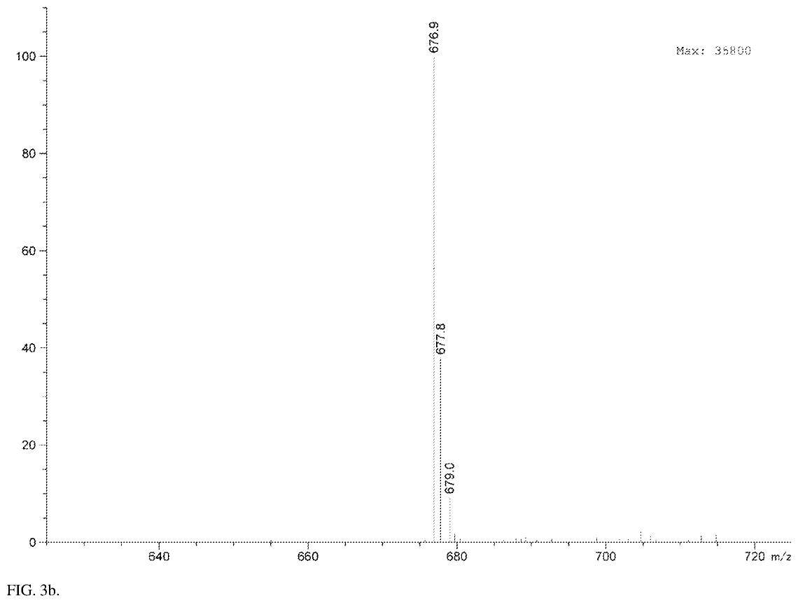

COSY spectrum with off-diagonal peaks are listed in Table 4a. The spectrum correlated many of the coupling patterns seen in the 1H spectrum, and further revealed that each trans-alkene is coupled to two aromatic protons, as indicated by weak 4J couplings. Furthermore, HMBC spectrum showed that each alkene is attached to one carboxyl group, thus revealing two caffeoyl groups, labelled with (′) and (″). The nine carbons of the caffeoyl groups are labelled 1-9 in accordance to common numbering system seen in the literature. HMBC further revealed that carboxyl 9′ is attached to quinic acid at C4 and carboxyl 9″ is attached to quinic acid at C5. The glucose unit is attached to caffeoyl (′) at position 4, as indicated by 3J HMBC from H1-Glc to C4′, and this glucosidic linkage is further supported by a 5J through-space ROESY interaction from H1-Glc to H5′. The anomeric center of glucose was assigned as alpha, due to the small coupling constant (d, 3.7 Hz) of H1-Glc. Altogether, the covalent linkages are established, consistent with the formula C₃₁H₃₄O₁₇ proposed by LCMS (see FIGS. 3a and 3b). The structure of Compound A is shown below.

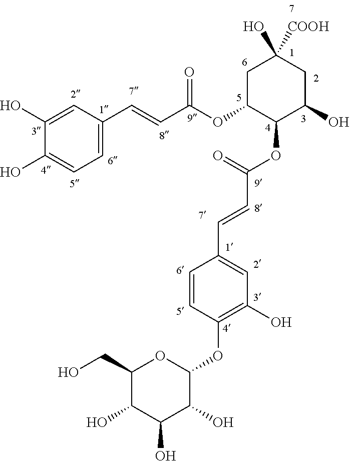 [SMILES: O=C(/C=C/c1ccc(O[C@H]2O[C@H](CO)[C@@H](O)[C@H](O)[C@H]2O)c(O)c1)O[C@@H]1[C@H](O)C[C@@](O)(C(=O)O)C[C@H]1OC(=O)/C=C/c1ccc(O)c(O)c1]

LCMS (FIGS. 3a and 3b) analysis of compound A showed a [M-H]⁻ ion at m/z 676.9, in good agreement with the expected molecular formula of C₃₁H₃₄O₁₇ (calculated for [C₃₁H₃₃O₁₇]⁻ monoisotopic ion: 677.2). The MS data confirms that compound A has a molecular formula of C₃₁H₃₄O₁₇. LCMS analysis was performed in the following conditions listed in Table 5.

TABLE 5
Conditions for LCMS analysis

| Column | Agilent Poroshell 120 SB-C18, 4.6 mm × 150 mm, | | |
|---|---|---|---|
| | 2.7 μm | | |
| Temperature | 40° C. | | |
| Mobile Phase | A: 0.1% (v/v) formic acid | | |
| | B: Acetonitrile | | |
| Gradient | Time (min) | A (%) | B (%) |
| | 0 | 90 | 10 |
| | 20 | 80 | 20 |
| | 25 | 70 | 30 |
| | 35 | 60 | 40 |
| | 40 | 60 | 40 |

| | |
|---|---|
| Flow rate | 1.0 mL/min |
| Injection | 5 μL |
| Run time | 40 mins |
| Post time | 10 mins |
| Autosampler | Ambient |
| temperature | |
| Detection | DAD and MSD at Negative Scan mode |
| DAD Settings | UV at 324 nm (4 nm bandwidth), Reference: |
| | 360 nm (100 nm bandwidth) |
| MSD Setting | Mode: ES-API, Negative Polarity |
| | Drying gas flow: 13.0 L/min |
| | Nebulizer Pressure: 30 psig |
| | Drying gas temperature: 270° C. |
| | Fragmentor: 50 V |
| | Scan ranges: 500 to 1500 of mass |
| Sample Preparation | 78 mg/L (30% ACN in water) |

### Example 5

Solubility of CGA and GCGA were tested in water, methanol and ethanol as described here. For CGA, about 0.1 g of Stevia CGA composition (see FIG. 2a) was mixed with the solvent slowly at room temperature, followed by incremental addition of solvent until the material was fully dissolved.

For GCGA, about 0.1 g the dried pellet from EXAMPLE 3 was mixed with the solvent slowly at room temperature, followed by incremental addition of solvent until the material was fully dissolved. Table 6 shows the solubility of CGA and GCGA in various solvents tested. As seen, GCGA has a significantly higher solubility than CGA in water.

TABLE 6
Solubility of CGA and GCGA in various solvents

| Sample | Solvent | Solubility, % w/v |
|---|---|---|
| CGA | Water | 0.5 |
| | Methanol | <0.1 |
| | Ethanol | <0.1 |
| GCGA | Water | 50 |
| | Methanol | <0.1 |
| | Ethanol | <0.1 |

### Example 6

Stability of CGA and GCGA at different pH were tested as described here. For CGA, about 50 mg of Stevia CGA composition (see FIG. 2a) was dissolved in 2 mL of buffer of pH 2. Two additional samples were prepared similarly in buffer of pH 6 and 9, respectively. This set of samples was incubated at room temperature.

Another set of three CGA samples was prepared in buffer of pH 2, pH 6 and pH 9 as described above and incubated at 4° C.

For GCGA, about 50 mg of the dried pellet from EXAMPLE 3 was dissolved in 2 mL of buffer of pH 2. Two additional samples were prepared similarly in buffer of pH 6 and 9, respectively. This set of samples was incubated at room temperature. Another set of three GCGA samples was prepared in buffer of pH 2, pH 6 and pH 9 as described above and incubated at 4° C.

The contents of CGA and GCGA throughout the duration of the study were analyzed by HPLC following the method in EXAMPLE 2. Data were presented as percentage of normalized peak area, calculated using the formula below.

$$Normalized peak area , \% = \frac{Peak area on a particular storage day}{Peak area on day 0} \times 100$$

The percentage indicates the percentage of CGA or GCGA compounds remained during storage. Therefore, the higher the percentage, the lesser the degradation of CGA or GCGA compounds during storage.

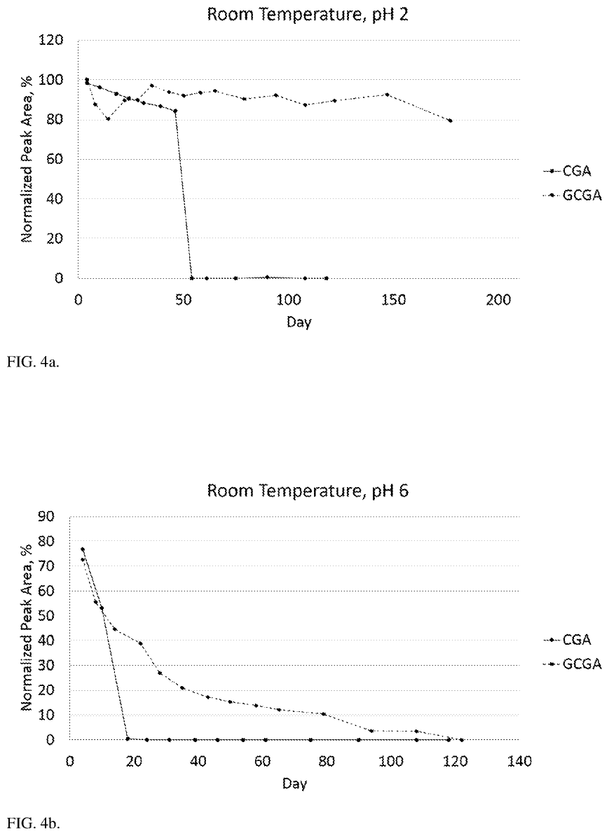

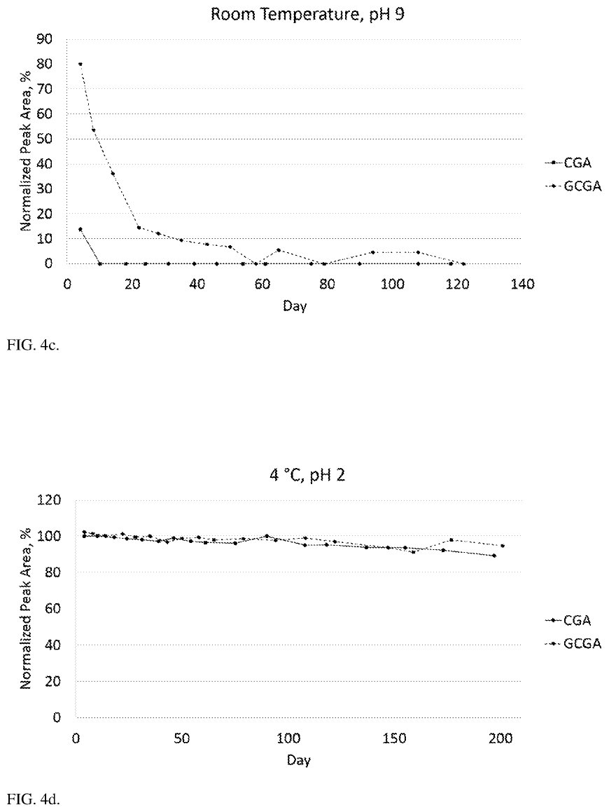

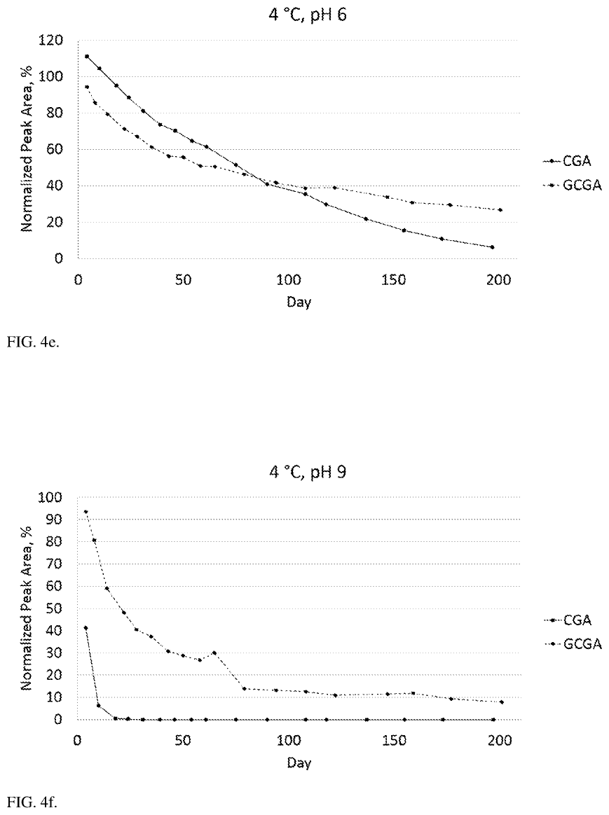

FIGS. 4a through 4c show that GCGA has better stability compared to CGA at pH 2, pH 6 and pH 9 at room temperature during storage. Among the samples at room temperature, GCGA at pH 2 has the highest stability with 80% of the compounds remained after storage for 177 days.

At 4° C., GCGA also showed better stability over CGA when stored at pH 2, pH 6 and pH 9 (see FIGS. 4d through 4f). At pH 2, GCGA and CGA did not have significant difference throughout the storage of around 170 days except until the last storage day whereby GCGA and CGA have 95% and 89% of compounds remained, respectively. At pH 6, GCGA showed slower degradation degree of the compounds although CGA was more stable than GCGA during the first 90 days of storage.

### Example 7

Stability of CGA and GCGA at pH 2, pH 2.5, pH 3, pH 3.5 and pH 4 were tested as described here. For CGA, about 50 mg of Stevia CGA composition (see FIG. 2a) was dissolved in 2 mL of buffer of pH of 2. Four additional samples were prepared similarly with in buffer of pH 2.5, pH 3, pH 3.5 and pH 4, respectively. This set of samples was incubated at room temperature.

Another set of five CGA samples was prepared at pH 2, pH 2.5, pH 3, pH 3.5 and pH 4 as described above and incubated at 4° C.

For GCGA, about 50 mg of the dried pellet from EXAMPLE 3 was dissolved in 2 mL of buffer of pH of 2. Four additional samples were prepared similarly with in buffer of pH 2.5, pH 3, pH 3.5 and pH 4, respectively. This set of samples was incubated at room temperature.

Another set of five GCGA samples was prepared at pH 2, pH 2.5, pH 3, pH 3.5 and pH 4 as described above and incubated at 4° C.

The contents of CGA and GCGA throughout the duration of the study were analyzed by HPLC following the method in EXAMPLE 2. Data were presented as percentage of normalized peak area, calculated using the formula below.

$$Normalized peak area , \% = \frac{Peak area on a particular storage day}{Peak area on day 0} \times 100$$

The percentage indicates the percentage of CGA or GCGA compounds remained during storage. Therefore, the higher the percentage, the lesser the degradation of CGA or GCGA compounds during storage.

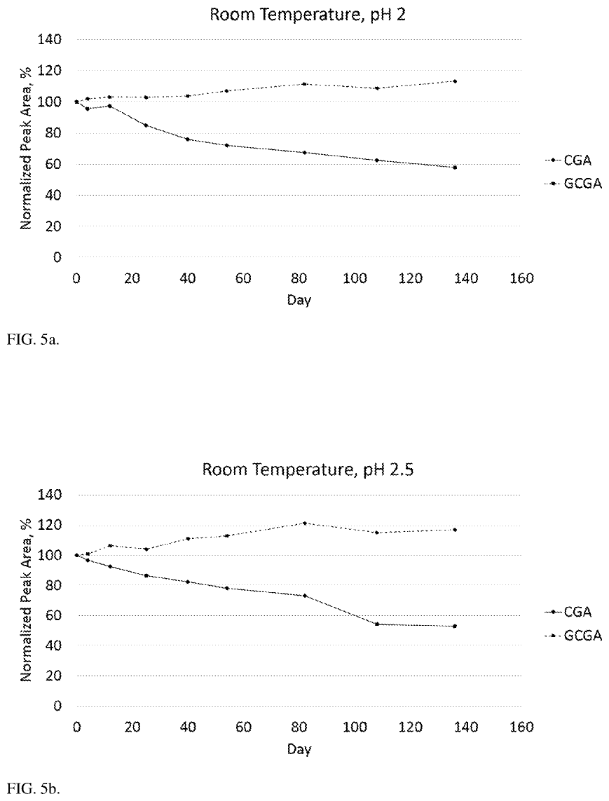

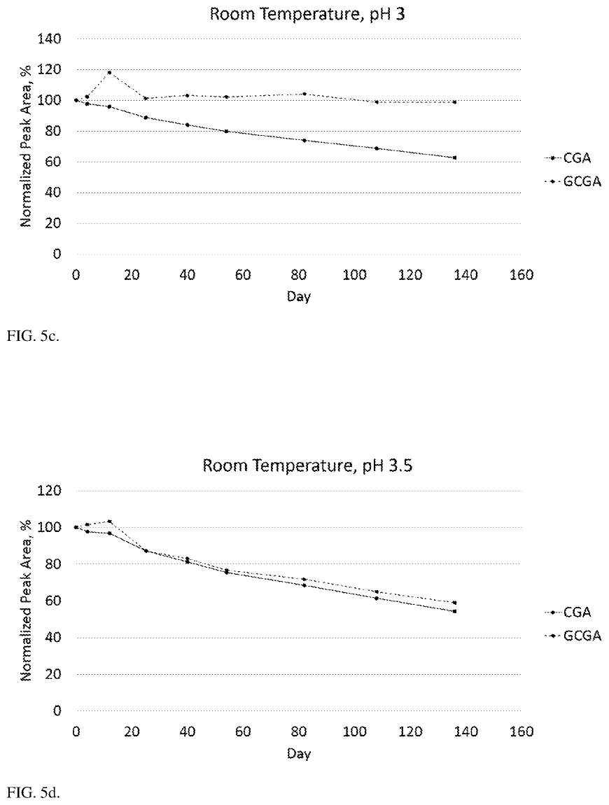

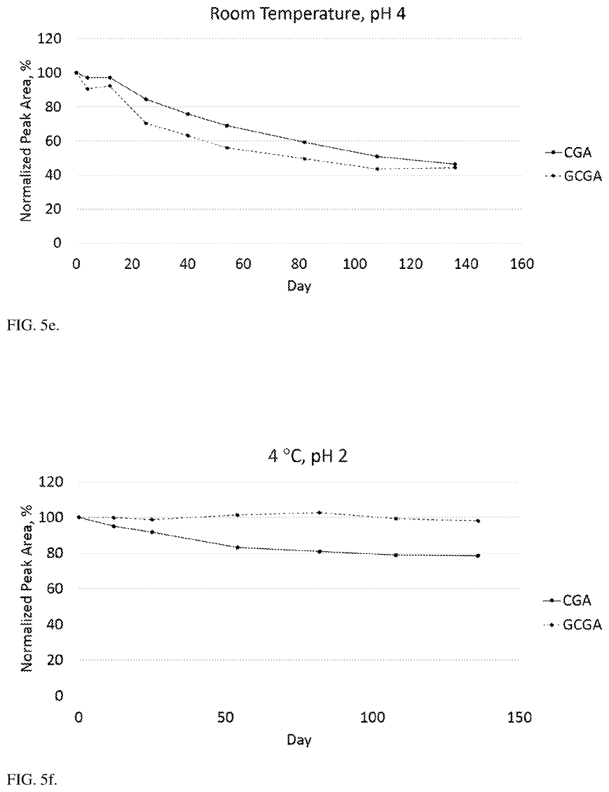

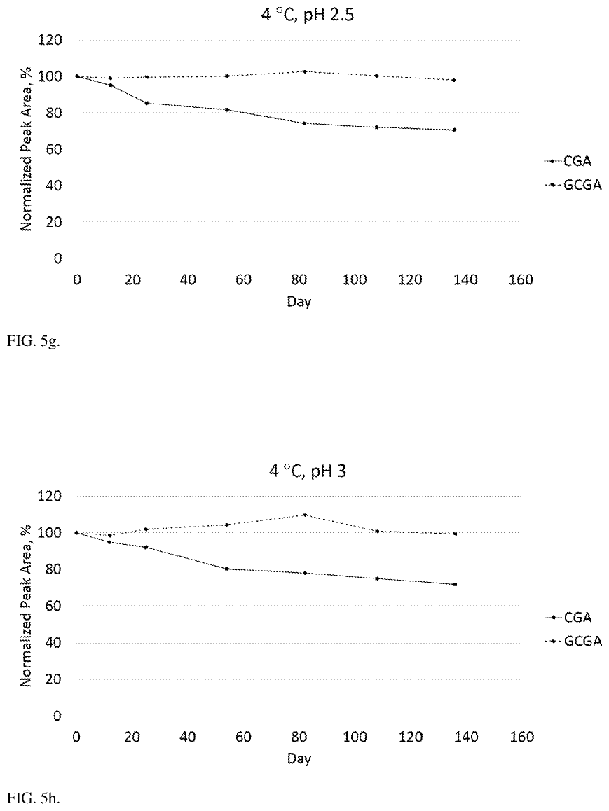

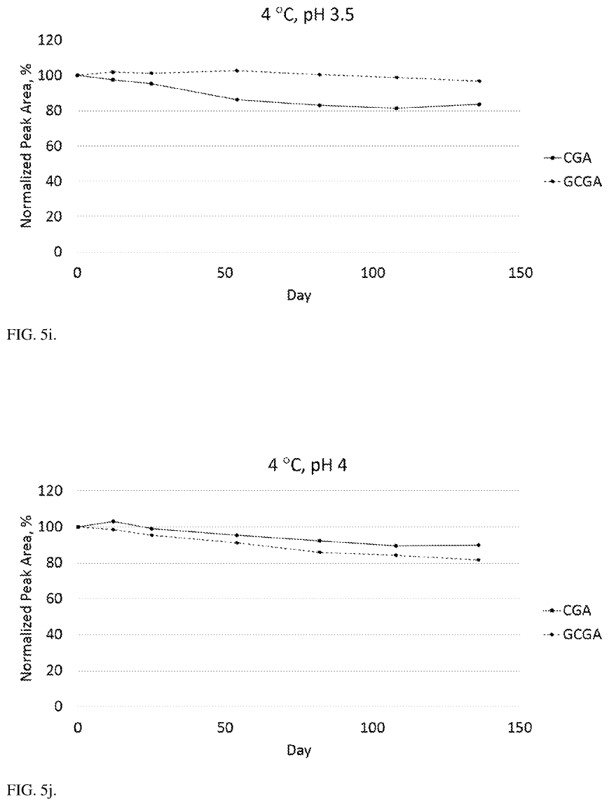

FIGS. 5a through 5j show the pH stability of CGA and GCGA at pH 2, 2.5, 3, 3.5 and pH 4 at room temperature and 4° C. At pH 2, pH 2.5, pH 3 and pH 3.5, GCGA has greater stability compared to CGA throughout the storage for both room temperature and 4° C. On the other hand, at pH 4, CGA has higher stability over GCGA throughout the storage for both room temperature and 4° C.

### Example 8

The dried pellet from EXAMPLE 3 was used to analyze its color stabilization function on blue pea flower extract solution. About 0.75 g of blue pea flower powder was mixed in 1 L water by stirring at room temperature for 30 minutes. The solution was filtered through Whatman filter paper. About 0.63 g sodium citrate and 1.51 g citric acid were added into the solution and mixed well until fully dissolved. Then, 2 g of potassium sorbate and 1 g of sodium benzoate were added into the mixture and mixed well until fully dissolved. The pH of the mixture was then adjusted to pH 3 using HCl. The mixture was divided into 200 mL for each flask. One flask was labeled as control while the other flask was added with 40 mg dried pellet from EXAMPLE 3. The samples were stored at room temperature with sunlight exposure for its color changes assessment. The absorbance of the solution throughout storage was measured at 576 nm. Data was reported as percentage of normalized absorbance, calculated using the formula below.

$$Normalized absorbance , \% = \frac{Absorbance on a particular storage day}{Absorbance on day 0} \times 100$$

The percentage indicates the remained percentage of color intensity of blue pea flower extract solution during storage. Therefore, the higher the percentage, the better the preservation of the color of blue flower extract solution during storage.

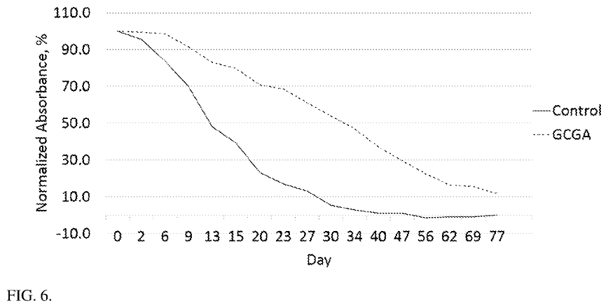

FIG. 6 shows the color stability of the blue pea flower extract solution throughout the storage at room temperature. Based on FIG. 6, the blue pea flower extract solution added with GCGA showed better color stability compared to the control over the storage period.

We claim:

1. A compound selected from compounds of structural formula (E) 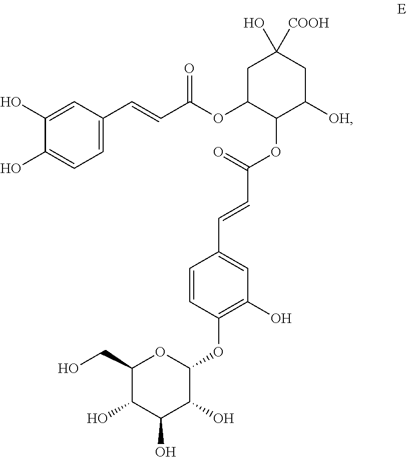 [SMILES: O=C(/C=C/c1ccc(O)c(O)c1)OC1CC(O)(C(=O)O)CC(O)C1OC(=O)/C=C/c1ccc(O[C@H]2O[C@H](CO)[C@@H](O)[C@H](O)[C@H]2O)c(O)c1]
salts thereof, and combinations thereof.

2.-9. (canceled)

10. A product comprising a compound of claim 1, wherein the product is selected from the group consisting of foods, beverages, pharmaceutical compositions, tobacco products, nutraceutical compositions, oral hygiene compositions, home care products, personal care products, and cosmetic compositions.

11. The product of claim 10, wherein the product is selected from the group consisting of alcoholic beverages, natural juices, refreshing drinks, carbonated soft drinks, diet drinks, zero calorie drinks, reduced calorie drinks and foods, yogurt drinks, instant juices, instant coffee, powdered types of instant beverages, canned products, syrups, fermented soybean paste, soy sauce, vinegar, dressings, mayonnaise, ketchups, curry, soup, instant bouillon, powdered soy sauce, powdered vinegar, types of biscuits, rice biscuit, crackers, bread, chocolates, caramel, candy, chewing gum, jelly, pudding, preserved fruits and vegetables, fresh cream, jam, marmalade, flower paste, powdered milk, ice cream, sorbet, vegetables and fruits packed in bottles, canned and boiled beans, frozen beef, frozen pork, frozen goat, frozen lamb, frozen mutton, frozen poultry like frozen chicken, frozen duck and frozen turkey, frozen venison, frozen fish, frozen crustaceans like frozen crab and frozen lobster, frozen molluscs like frozen clams, frozen oysters, frozen scallops, and frozen mussels, frozen shrimps, frozen octopus, frozen squid, fresh beef, fresh pork, fresh goat, fresh lamb, fresh mutton, fresh poultry like fresh chicken, fresh duck and fresh turkey, fresh venison, fresh fish, fresh crustaceans like fresh crab and fresh lobster, fresh molluscs like fresh clams, fresh oysters, fresh scallops, and fresh mussels, fresh shrimps, fresh octopus, fresh squid, meat and foods boiled in sweetened sauce, agricultural vegetable food products, seafood, ham, sausage, fish ham, fish sausage, fish paste, deep fried fish products, dried seafood products, frozen food products, preserved seaweed, preserved meat, tobacco, lipsticks and medicinal products.

12. The product of claim 10, wherein the product is selected from the group consisting of laundry detergents, fabric conditioners, dishwashing detergents, hard floor and surface cleaners, glass cleaners, carpet cleaners, oven cleaners, air fresheners, car shampoo, toilet care products, and insect-control products.

13. The product of claim 10, wherein the product is selected from the group consisting of lotions, cosmetics, hair dyes, hair creams, face creams, body creams, deodorants, sunscreens, perfumes, toothpastes, bath soaps, hand soaps, shampoos, conditioners, hair masks, hair sprays, hair treatment oils, face cleansers, face masks, face moisturizers, acne creams, nail polishes, nail polish removers, shaving creams, shaving foams, shaving gels, after-shave lotions, after-shave balms, after-shave creams, after-shave fluids, shower gels, shower liquids, body sprays, anti-perspirants, skin balms, skin wipes, baby oils, baby colognes, baby shampoos, baby body shampoos, baby lotions, baby bath liquids, baby soaps, baby gel lotions, baby head-to-toe baths and baby powders.

14. The product of claim 10, further comprising at least one additive selected from the group consisting of steviol glycosides, carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, caffeine, flavorants and flavoring ingredients, astringent compounds, proteins or protein hydrolysates, surfactants, emulsifiers, flavonoids, alcohols, polymers, salts thereof and combinations thereof.

15. The product of claim 10, further comprising at least one functional ingredient selected from the group consisting of antioxidants, saponins, dietary fiber sources, fatty acids, vitamins, glucosamine, minerals, preservatives, hydration agents, probiotics, prebiotics, weight management agents, osteoporosis management agents, phytoestrogens, long chain primary aliphatic saturated alcohols, phytosterols, salts thereof, and combinations thereof.

16. The product of claim 10, further comprising a compound selected from the group consisting of vitamin C, vitamin E, carotenoid, flavonols, flavanols, flavanones, flavones, isoflavonoids, anthocyanidins, glingerol, hydroxycinnamic acid, hydrobenzoic acid, curcumin, gallic acid, rosemary extract, rosmarinic acid, caffeic acid, carnosic acid, carnosol, butylated hydroxytoluene (BHT), butylated hydroxyanisole (BHA), tert-butylated hydroxyquinone (TBHQ), propyl 3,4,5-trihydroxybenzoate (PG), and 1,2,3-trihydroxybenzene (Pyrogallol-PY), salts thereof, and combinations thereof.

17. A method for increasing the stability of a product towards oxidation, comprising: adding a compound of claim 1 to a product selected from the group consisting of beverage products, food products, and home care products, wherein the compound is present in an amount ranging from about 0.005% to about 0.5% by weight, to generate a product having improved stability towards oxidation throughout storage compared to a control product that does not contain the compound.

18. A method for stabilizing the color of a product, comprising: adding a compound of claim 1 to a product selected from the group consisting of beverage products, food products, and home care products, wherein the compound is present in an amount ranging from about 0.005% to about 0.5% by weight, to generate a product having improved color stability compared to a control product that does not contain the compound.

19. A method for stabilizing the flavor of a product, comprising: adding a compound of claim 1 to a product selected from the group consisting of beverage products, food products, and home care products, wherein the compound is present in an amount ranging from about 0.005% to about 0.5% by weight, to generate a product having improved flavor stability compared to a control product that does not contain the compound.

20. A method for modifying a flavor profile of a product, comprising: adding a compound of claim 1 to a product selected from the group consisting of beverage products, food products, and home care products, wherein the compound is present in an amount ranging from about 0.005% to about 0.5% by weight, to modify the flavor profile of the product without imparting detectable sweetness or flavor to the product.

21. A method for suppressing foaming of a product, comprising: adding a compound of claim 1 to a product selected from the group consisting of beverage products, food products, and home care products, wherein the compound is present in an amount ranging from about 0.005% to about 0.5% by weight, to generate a product with suppressed foaming.

22. A method for enhancing the solubility of insoluble material in a product, comprising: adding a compound of claim 1 to a product selected from the group consisting of beverage products, food products, and home care products, wherein the compound is present in an amount ranging from about 0.005% to about 0.5% by weight, to generate a product having less insoluble material compared to a control product that does not contain the compound.

23. A method for modifying the fragrance of a product, comprising: adding a compound of claim 1 to a product selected from the group consisting of beverage products, food products, and home care products, wherein the compound is present in an amount ranging from about 0.005% to about 0.5% by weight, to generate a product having a modified fragrance compared to a control product that does not contain the compound.

24. A method for stabilizing the fragrance of a product, comprising: adding a compound of claim 1 to a product selected from the group consisting of beverage products, food products, and home care products, wherein the compound is present in an amount ranging from about 0.005% to about 0.5% by weight, to generate a product having enhanced fragrance stability compared to a control product that does not contain the compound.

25. A method for enhancing the fragrance of a product comprising: adding a compound of claim 1 to a product selected from the group consisting of beverage products, food products, and home care products, wherein the compound is present in an amount ranging from about 0.005% to about 0.5% by weight, to generate a product having enhanced fragrance compared to a control product that does not contain the compound.

26. A method for deodorizing a product comprising: adding a compound of claim 1 to a product selected from the group consisting of beverage products, food products, and home care products, wherein the compound is present in an amount ranging from about 0.005% to about 0.5% by weight, to generate a product with less or no odor compared to a control product that does not contain the compound.
